(19) United States
(12) Patent Application Publication
(10) Pub. No.: US 2026/0183814 A1
(43) Pub. Date: Jul. 2, 2026

(54) Waste Management Using Biotic and Abiotic Compositions

(71) Applicant: Hiro Technologies Inc., Austin, TX (US)

(72) Inventors: Miki Agrawal, Austin, TX (US); Tero Isokauppila, Austin, TX (US); Danielle Stevenson, Austin, TX (US); Elizabeth Bowman, Round Rock, TX (US); Sarah Larocca, Austin, TX (US); Lowry Mays, Austin, TX (US); Eric Ricciardi, Austin, TX (US); Wade Hubbard, Fort Mill, SC (US); David Gonzalez, Austin, TX (US); Nicholas Romano, Austin, TX (US)

(21) Appl. No.: 18/835,522

(22) PCT Filed: Jun. 9, 2024
(86) PCT No.: PCT/US2024/033176

§ 371 Date: Aug. 2, 2024

Related U.S. Application Data
(60) Provisional application No. 63/507,180, filed on Jun. 9, 2023.

(51) Int. Cl. B09B 3/60; B09B 101/75; C12N 1/16; C12N 11/00; C12R 1/645

(52) U.S. Cl. CPC B09B 3/60; C12N 1/16; C12N 11/00; B09B 2101/75; C12R 2001/645

(57) ABSTRACT

An inoculant composition has a first fungal species or strain and a different second fungal species or strain. At least one of the first fungal species or strain is a saprophytic fungi. Each of the first and second fungal species or strain is present in a relative concentration that within the composition to allow inoculation on one or more polymer materials at an inoculation ratio of no greater than 1:1 fungal biomass to polymer material mass. Additionally, an inoculant can include a first engineered fungal species or strain that recognizes plastic products as a food source and digest recalcitrant long-chain carbons more efficiently than a naturally occurring counterpart of the same species or strain. The first engineered fungal species or strain may be immobilized rendering it dormant from metabolism. A method for transforming long chain carbon materials in plastic waste includes adding an inoculum to a plastic substrate.

### BACKGROUND

The present invention relates generally to the field of degradation and, more specifically, to compositions of matter and methods for enhancing digestion of recalcitrant long chain carbon materials, such as plastics, as well as other materials found alongside plastic waste products.

In recent years, the negative impact of plastic waste on the environment has become a growing concern. Traditional plastic materials can take hundreds of years to degrade, leading to environmental pollution and ecological damage. The present invention addresses this problem by enhancing the digestion of plastic and other synthetic or organic materials.

The present invention provides methods for enhancing the digestion of said materials. The methods involve the use of pre-selected fungal species or strains that have been trained to digest recalcitrant polymers. The selected fungal species are immobilized in a manner that maximizes efficiency, effectiveness, and ease of use, and can be combined in a mixture of fungal species to achieve the desired digestion result.

When fungi are inoculated in a mixture with plastic materials present, they first grow and spread on the surface of the material using their mycelium, which is a network of thread-like structures that make up the body of the fungus. This growth and spreading of the mycelium is called mycelial growth. During mycelial growth, the fungi secrete various enzymes that can break down different types of compounds in the plastic material, such as cellulose, and lignin.

The present invention relates to the field of fluid soft plastics such as films and nonwovens, and their product compositions such as fluid absorbent articles, particularly to the field of diapers and other personal waste disposal items. The invention pertains to the development of novel absorbent materials and constructions that can be used in a variety of absorbent products. The aim of performance of absorbent products is to provide improved absorbency, fluid retention, and overall performance, while also ensuring comfort, fit, and ease of use. Methods provided do not require new materials in order to make a more sustainable product, eliminating the tradeoff of these performance vectors and sustainability.

An absorbent article, such as a diaper, is typically composed of several layers of materials designed to manage and contain human or animal excrement. The layers in a diaper can include a topsheet, an acquisition layer, an absorbent core, a backsheet, and various other materials such as adhesives, elastics, and fasteners.

The topsheet layer is typically a nonwoven material made from PE, PP, bico, or other polymers, and is designed to be in contact with the skin. The acquisition layer is designed to quickly wick away liquid from the skin and into the absorbent core. The absorbent core is typically composed of cellulose fibers and a superabsorbent polymer (SAP) to capture and hold liquid.

The backsheet layer is typically a film made from PE, PP, or other polymers, and is designed to prevent liquid from leaking out of the diaper. Other materials found in diapers can include hot melts for adhesive purposes, surfactants and dyes for wetness indicators, and various fastening components such as Velcro and elastics.

The different types of polymers found in diapers serve specific purposes in the diaper's design, such as absorbency, liquid management, and containment. SAP is a key polymer found in diapers, as it can absorb up to several hundred times its weight in liquid, making it highly effective in capturing and holding liquid.

### BRIEF DESCRIPTION OF THE DRAWINGS

FIG. 1A shows the chemical structure of some of the most common plastics as background to the disclosed embodiments;

FIG. 1B is a graphical equation showing that embodiments of the disclosed inoculum is effective at inoculating plastic material at a weight ratio below 1:1 inoculum:plastic;

FIG. 2 depicts colorimetric analysis demonstrating lignin peroxidase from Species S, and laccase and manganese peroxidase [C] production from Species W;

FIG. 3 show SEM images showing the efficacy of the disclosed embodiments in Example 3;

FIG. 4 are SEM images showing that BSNW was effectively inoculated with Species W in Example 3;

FIG. 5 includes TGA plots showing efficacy of the disclosed embodiments in Example 3;

FIGS. 6-8 include DSC plots showing efficacy of the disclosed embodiments in Example 3;

FIG. 9 includes FTIR plots showing efficacy of the disclosed embodiments in Example 3;

FIG. 10 shows an exemplary adaptation process according to the disclosure;

FIG. 11 includes carbonyl index plots of embodiments of the disclosure showing digestion efficacy;

FIG. 12 includes FTIR plots showing significant differences between performance of adapted species according to the disclosure and non-adapted controls of the same species;

FIG. 13 shows an exemplary adaptation process according to the disclosure;

FIG. 14 is a graphical equation showing that the disclosed embodiments of the inoculum with engineered fungal species are effective at inoculating plastic waste in disposal conditions with other waste including natural polymer waste at an inoculation rate less than 1:1 fungal inoculant to waste;

FIGS. 15-16 include FTIR data showing growth area on a diaper under different conditions for species according to the disclosure;

FIG. 17 are photographs showing fungal growth on diapers with embodiments of disclosed inoculant;

FIG. 18 includes FTIR data comparing embodiments of species adapted according to the disclosure with non-adapted counterpart species;

FIG. 19 graphically depicts embodiments of the disclosed methods;

FIG. 20 graphically depicts embodiments of species consortia of the disclosure for degrading waste;

FIG. 21 includes photographs showing growth of each of the inoculum blends of Example 8;

FIG. 22 includes photographs of species blends on petri dishes according to Example 8;

FIG. 23 shows carbonyl index data for samples of multiple engineered species separately and together;

FIG. 24 shows emergence data from beads according to the disclosure and Example 8;

FIG. 25 graphically depicts various form factors applicable to the disclosed embodiments;

FIG. 26 is a graphical equation showing a dry inoculum combined with contaminated polymer products at a low inoculation rate in the presence of moist contaminants;

FIG. 27 is a photograph showing emergence of fungi from beads of immobilized encapsulated mycelium in an exemplary waste product environment according to Example 10;

FIG. 28 are photographs of petri dishes showing emergence of nonwoven adhered immobilize fungal inoculum according to Example 12;

FIG. 29 includes photographs showing the rehydration process and emergence of inoculums according to the disclosure;

FIG. 30 includes photographs showing emergence and vigorous colonization of diaper materials by fungal hyphae according to the disclosure;

FIG. 31 graphically depicts a process for growing filamentous fungi on polymer scaffolds for forming a shelf-stable product according to the disclosure;

FIG. 32 includes photographs showing emergence of fiber scaffold inoculum of an engineered species according to the disclosure;

FIG. 33 includes photographs showing emergence of enrobed fiber inoculum of an engineered species according to the disclosure;

FIG. 34 graphically depicts a method of making an absorbent product according to the disclosure;

FIG. 35 includes photographs showing emergence of fungal inoculum from engineered species from an absorbent product according to the disclosure;

FIG. 36 includes photographs showing emergence of a fungal inoculum from a film container according to the disclosure;

FIG. 37 includes photographs showing results of Example 15;

FIG. 38 is a photograph showing absorbency results of Example 16;

FIG. 39 graphically depicts the unexpected synergistic absorbance effect of combining a natural hydrogel and synthetic hydrogel; and

FIG. 40 is a graphical depiction of a plastic waste fungal inoculum treatment regime according to the disclosed embodiments.

### SUMMARY

Plastic pollution has become a global issue impacting almost every ecosystem. Due to their utility, widespread usage, and ultimate impact of plastic polymers, research into the degradation of plastics has become a promising field. There are a wide variety of plastic polymers in use that are variable in their chemical composition requiring a parallel variety of enzymes to achieve complete breakdown into microbial biomass, water, and carbon dioxide.

Rates of plastic degradation are dependent on traits of individual plastics, such as hydrophobicity, chemical structure, degrees of crystallinity, and molecular weight. Higher molecular weight plastics and plastics with a higher proportion of crystalline regions are more recalcitrant to digestion and degradation. The relationship of chemical structure to degradation, though, is less linear. Broadly, there are two categories of plastic polymers: those composed of repeated chains of the same chemical elements (referred to herein as homochains) and those composed of a more heterogeneous mixture of elements (referred to herein as heterochains). Homochain polymers such as polyethylene (PE) and polypropylene (PP) are more resistant to microbial degradation compared to heterochains such as polyurethane (PU), polyethylene terephthalate (PET), and polystyrene (PS). PET and PU are the two most deeply studied types of plastics, although the most common plastics produced annually are PE (30%) and PP (19%)
indicating that more research into degradation pathways is needed. The examples provided herein for heterochains, homochains, and crosslinked polymers are illustrative and serve to demonstrate the various digestion mechanisms specific to each polymer family. These nonlimiting examples are categorized based on their respective digestion mechanisms, and as those skilled in the art will readily understand, the methods described are applicable to a broader range of materials within these polymer families.

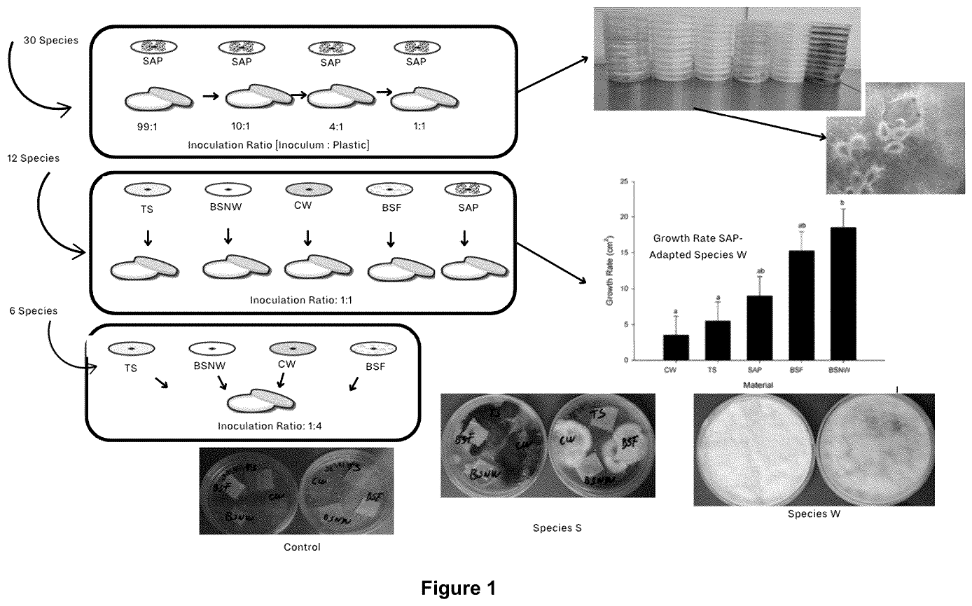

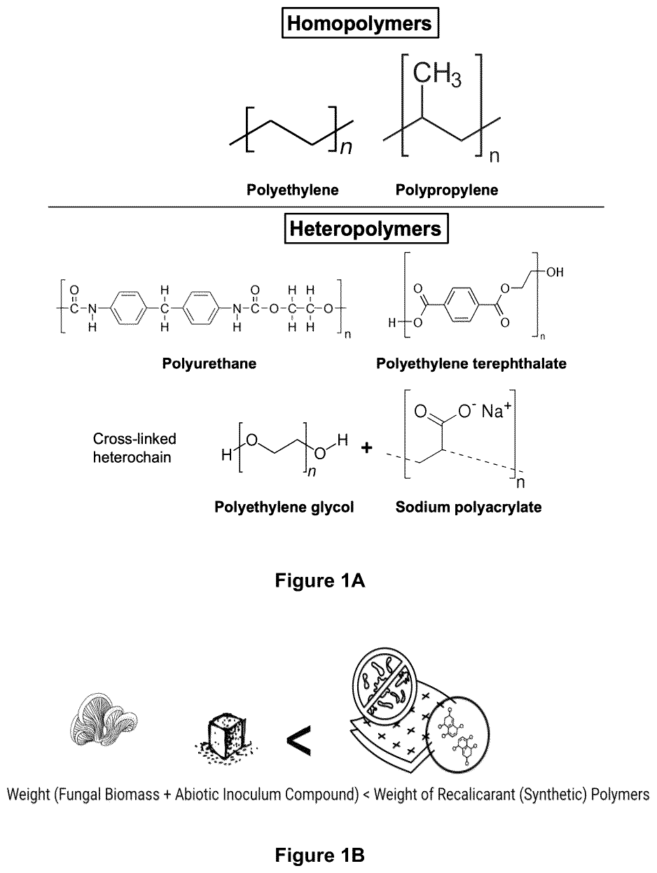

By way of background, FIG. 1A shows the chemical structure of some of the most common plastics. These are the main plastics found in diapers. Cross-linked heterochain composed of polyethylene glycol (PEG) and sodium polyacrylate (NaPA) are the components of super absorbent polymers which are found in all absorbent products. The wide variation in chemical composition is one reason that mixed types of plastics cannot be recycled together.

Generally, homochains are polymers with backbones made up of carbon and hydrogen atoms exclusively. This simplicity in structure suggests a uniform bonding pattern, focusing on carbon-carbon linkages. For enzymatic digestion, homochains require an initial oxidation process before hydrolysis can occur. The absence of heteroatoms in their backbone makes direct hydrolysis less feasible, necessitating an oxidation step to introduce functional groups for enzymatic attack. Oxidation can occur through abiotic (e.g. heat or chemical) or biological means. Given their composition and digestion requirements, it is believed that homochains are inherently more resistant to digestion, potentially extending the time needed for complete breakdown.

Heterochains are characterized by the presence of oxygen and other non-carbon, non-hydrogen elements, such as sodium (Na) and nitrogen (N) in the polymer backbone, for example. This diversity introduces a variety of functional groups and bonding arrangements within the polymer structure. The presence of oxygen and other heteroatoms allows for hydrolysis at the point of inoculation without the need for prior oxidation. This structural complexity can facilitate enzymatic access and attack, potentially streamlining the digestion process. It is believed that heterochains may degrade more readily than homochains due to their varied and less uniform structure.

Distinct assemblages of fungi colonize plastic (i.e. plastic as a substrate acts as a community filter); these microbial communities have been termed the plastisphere. What aspects of the plastisphere act as community filters and what specific traits allow fungi to overcome this barrier is not well understood. Certain fungal traits facilitate plastic colonization and digestion. Hydrophobin production assists in the colonization of the hydrophobic surfaces of plastics through the formation of aerial structures. Hydrophobins enable hyphal attachment to hydrophobic plastic surfaces with evidence indicating that some can dramatically increase hydrolysis. Exopolysaccharide production further assists with adhesion to substrate surfaces creating a fungal-substrate interface for enzyme secretion. Most of the research into plastic degradation and/or digestion has been in highly controlled lab settings with “pristine” and/or pre-treated plastics (i.e. no additives or differences in surface texture), rather than consumer-grade plastic products under natural conditions limiting real-world inferences that can be made. Overall, fungi show diverse enzymatic capabilities to digest and/or degrade conventional plastics, but there remains a need for an effective method of increasing the rate of digestion of commercial grade plastic waste.

The disclosed embodiments provide a method for degrading various plastic waste products, including absorbent articles, using preselected fungi capable of breaking down recalcitrant long-chain carbons, both organic and synthetic. The embodiments provide for the widespread commercialization of the method within the intended fields of use, such as diapers and absorbent products. The fungi can be stored in a shelf-stable form and added to plastic-containing products during manufacturing or after use, promoting environmental sustainability by reducing plastic waste.

Also provided is a composition of matter that includes an immobilized, shelf-stable fungal inoculant, which has been trained to target and digest polymers found in polymer-based materials. This composition, comprising one or more fungal species or strains, may be encapsulated within an immobilization material, ensuring its stability, protection from contamination, and facilitating its shelf-stable nature. Upon activation through moisture exposure, the encapsulated fungi are capable of resuming their metabolism and promoting the digestion process, offering an eco-friendly solution for the digestion of various polymer-based materials.

Also provided herein is a method for the industrial-scale digestion of polymer-based materials, with an option to include organic waste, utilizing preselected fungal inoculants.

Waste materials are collected and aggregated from various streams, and the fungal inoculants are introduced. The environmental conditions within an industrial-scale waste processing system, such as a bioreactor, are controlled to optimize the fungal activity. Optional process aids can be introduced to enhance the growth and colonization of the rehydrated fungal inoculant. The digestion process results in the generation of biomass, water, carbon dioxide, and other byproducts. The resulting biomass can be utilized for further applications such as composting, soil amendment, or as feedstock for renewable energy production. The method provides an efficient, environmentally-friendly, and scalable approach to managing polymer-based and organic waste at an industrial scale.

In another embodiment, a method includes incorporating immobilized fungal inoculants into a product either before or after its usage. The fungal inoculants are configured to enable efficient digestion of the product at a significantly increased rate. The inoculation process is flexible, allowing consumers to apply the inoculant in various ways such as directly onto the used product, into a waste receptacle, or into a specialized digestion bag. The inoculant can also be incorporated during the manufacturing process of the product.

In another embodiment, a composition of matter comprises a mixture of adapted fungal species and strains, assembled into a plurality of encapsulated fungal inoculants or other immobilization materials, with each bead containing a single species or strain of fungus. The mixture of fungal species creates a novel “consortium” not found in nature, that promotes enhanced fungal growth and plastic digestion and exhibits resilience to abiotic stress, including feces, urine, and other conditions. The fungal species are trained to recognize plastic products as a food source and digest recalcitrant long-chain carbons found in synthetic polymers more efficiently than in their naturally occurring forms.

Also provided herein is a composition of matter comprising an immobilized, shelf-stable fungal inoculant, which has been trained to target and digest polymers found in polymer-based materials. This composition, comprising one or more fungal species or strains, is encapsulated within an immobilization material, ensuring its stability, protection from contamination, and facilitating its shelf-stable nature. Upon activation through moisture exposure, the encapsulated fungi are capable of resuming their metabolism and promoting the digestion process, offering an eco-friendly solution for the digestion of various polymer-based materials.

The species used in the disclosed embodiments are selected from white rot, brown rot and soft rot categories (Species W, B, S, respectively). Categorization of these species and their strains is based on attributes observed in nature, their morphology, and their genomic potential. Only a small fraction of the enzymatic potential of these fungal species in degrading plastics has been characterized previously. Therefore, the process of screening and adapting these fungi can upregulate the gene expression of known enzymes and activate the expression of enzymes not yet characterized. This complex mixture of enzymes from single species and multiple species enhances the digestion of complex polymers. This process involves iterative methodologies, including experimental optimization and systematic manipulation. The process of selecting, adapting, and combining these fungal species, which is measured by the experimental characterization of digestion markers on the polymer substrates, forms the basis of the inventive embodiments disclosed herein.

Also provided herein is a method for managing the rehydration and activation of a dormant, immobilized fungal inoculant within absorbent products. The process ensures optimal fungal performance, enhancing the product's biodegradability while maintaining its absorbency. The fungal inoculant's emergence is carefully controlled, allowing it to effectively digest the absorbent product post-use, contributing to a more sustainable waste management solution.

Also provided herein is a method for encapsulating and immobilizing preselected fungi, either in discrete beads or on a continuous substrate, facilitating their use for the digestion of recalcitrant long chain carbon materials. The process comprises homogenizing and optionally filtering fungal hyphae, mixing them with an alginate polymer solution, forming the mixture into beads or a hydrogel, and applying dehydration treatments. The encapsulated fungi can be coated for added protection and extended shelf life, with applications in various environments for digestion.

In another embodiment, a method comprises using immobilized fungal inoculants, which are incorporated into a product either before or after its usage, and are configured to enable efficient digestion of the product within improved time frames, preferably such as 6-12 months, for example (non-limiting). The inoculation process is flexible, allowing consumers to apply the inoculant in various ways such as directly onto the used product, into a waste receptacle, or into a specialized digestion bag. The inoculant can be incorporated during the manufacturing process of the product.

Also provided herein is a superabsorbent hydrogel embedded with a fungal inoculant, designed for use in absorbent products. The hydrogel exhibits high swelling capabilities, swift hydration rates, and excellent fluid retention capacity, while also providing the unique feature of a bioactive fungal inoculant. This combination results in an absorbent product with enhanced fluid handling properties and the added benefit of improved biodegradability, contributing to more sustainable and environmentally friendly absorbent products.

Embodiments of a method for incorporating a fungal inoculant into the manufacturing process of absorbent products to enhance their biodegradability is also provided. In one embodiment, the fungal inoculant is prepared as particles similar in size to superabsorbent polymers (SAPs). These particles are mixed with a pulp fiber mixture in a hopper, then formed into the absorbent core of the product. This process is designed to ensure the majority of the fungal inoculant particles do not reach temperatures that would kill the fungi in stasis.

In an alternative embodiment, the fungal inoculant is embedded in a nonwoven substrate, cut into patches, and applied to a partially assembled absorbent product using a vacuum rotating drum. The patches are secured with a tackifier, adhesive, or mechanical bonding and covered with a further layer for protection.

Also provided herein is a method for industrial-scale digestion of polymer-based materials, optionally including organic waste, utilizing preselected fungal inoculants. In this embodiment, waste materials are collected and aggregated from various streams, and the fungal inoculants are introduced. The environmental conditions within an industrial-scale waste processing system, such as an anaerobic or aerobic digestor or bioreactor landfill, are controlled to optimize the fungal activity. Optional process aids can be introduced to enhance the growth and colonization of the rehydrated fungal inoculant. The digestion process results in the generation of biomass, water, carbon dioxide, and other byproducts.

The resulting biomass can be utilized for further applications such as composting, soil amendment, or as feedstock for renewable energy production. This novel method provides an efficient, environmentally-friendly, and scalable approach to managing polymer-based and organic waste at an industrial scale.

### DETAILED DESCRIPTION

The digestion of a polymer can be described using a first-order kinetic model, which relates the rate of degradation to the remaining amount of material through an exponential decay equation:

C=C0*e\hat{ }(−kt)
wherein C is the concentration of remaining material, C0 is the initial concentration, k is the degradation rate constant, and t is the time.

Based on experimental studies, the estimated degradation times for some common polymer materials are as follows:
Superabsorbent polymers: these polymers are designed to be highly stable and can take hundreds of years to degrade. For example, a study found that superabsorbent polymers in soil had a half-life of about 300 years.
Thin PE film: the estimated degradation time for thin PE film in the environment is several hundred years. A study on the degradation of PE films in seawater found that after 28 months, the weight loss of the films was less than 3%.
PE nonwovens: like thin PE films, PE nonwovens can take several hundred years to degrade in the environment. A study on the degradation of nonwoven fabrics found that after 90 days, the weight loss of a PE nonwoven was less than 2%.
PP nonwovens: PP nonwovens can also take several hundred years to degrade. A study on the degradation of PP nonwovens in soil found that after 180 days, the weight loss of the materials was less than 5%.
Cellulose fiber: cellulose fiber is biodegradable and can degrade much faster than synthetic polymers, typically taking a few months to a few years depending on the conditions. A study on the degradation of cellulose fibers in soil found that after 90 days, the weight loss of the fibers was about 30%.

As used herein, “immobilize” means to fix fungi or bacteria in place within a material to facilitate targeted and controlled digestion. As used herein, “shelf stable” means an inoculant prepared in a way that remains viable over time without the need for refrigeration or other specific storage conditions. As used herein, “web conversion stable” means an inoculant that maintains viability throughout the manufacturing process of absorbent products, even under stress conditions like high temperatures. As used herein, “rehydrate” means a process by which dehydrated fungal inoculants absorb moisture to reactivate their metabolic processes for digestion. As used herein, “emerge” means a process by which fungal inoculants begin escaping from encapsulation, begin reactivation and grow and recognize the polymer matrix after rehydration or under conducive environmental conditions. As used herein, “fungi” refers to microorganisms used in the digestion process to break down polymer-based materials. As used herein, “preselected” refers to fungi chosen and screened based on their ability to digest specific types of polymer materials, in disposal environments, in presence of and effectively compete against biological and chemical contaminants. As used herein, “adapted” or “engineered” refers to fungal strains or microbial communities that have been genetically or environmentally conditioned to improve their digestion capabilities on specific substrates. As used herein, “colonize” refers to the ability of fungi or microbes to establish and multiply on polymer-based materials, initiating the digestion process. As used herein, “degrade” refers to the biochemical process by which fungi or microbes breakdown complex polymer chains into simpler, less harmful substances. As used herein, “waste stabilization” refers to techniques used to manage waste by minimizing the leaching of toxins and microplastics into the environment. As used herein, “enzymatic digestion” or “enzymatic degradation” refer to the process by which fungi produce a suite of enzymes (and other metabolites) that chemically break down polymers into simpler compounds. As used herein, “metabolize” refers to the process by which fungi absorb and convert carbon from polymers into energy and cellular materials during digestion. As used herein, “scalable” refers to a process or technology that can be expanded from lab-scale to industrial applications without losing functionality or efficacy. As used herein, “consortia” means a group of different microbial species that work together to digest polymers more efficiently than individual species alone. As used herein, “consortia composition” means the specific makeup of microbial consortia, often measured as the percentage of each species within the group. As used herein, “contaminate” refers to targeting contaminated or waste products, particularly those containing biowaste, for digestion. As used herein, “disposal environment” means a settings or conditions under which polymer-based products are disposed of and subjected to digestion processes. As used herein, “inoculum” refers to active fungal or microbial cells introduced to the polymer material to initiate digestion. As used herein, “inoculation rate” refers to the density of microbial or fungal cells introduced per unit weight or volume of substrate, critical for ensuring effective and scalable digestion. “As used herein, “inoculate” refers to an act of introducing microbial or fungal inoculants into polymer materials to start the digestion process. As used herein, “absorbency” (regarding a fluid) refers to the capacity of a material to absorb and retain fluids, relevant in the context of designing polymer-based materials like diapers that are targeted for digestion. As used herein, “retention” (regarding a fluid) refers to an ability of a product to hold absorbed fluid under pressure, crucial for the performance of products like sanitary pads and diapers. As used herein, “permeable” (regarding a fluid) describes materials that allow fluids to pass through, which can influence the plastic consumption rate and efficiency of microbial inoculants. As used herein, “integration” refers to the process of incorporating fungal inoculants into the polymer matrix during or after manufacturing to facilitate digestion. As used herein, “embed” refers to a process that involves integrating the fungal inoculants directly into the structure of the polymer-based material to ensure close contact and effective digestion. As used herein, “controlled time release” refers to a formulation feature that allows the timed release of fungal inoculants from a product to synchronize digestion with waste management schedules. As used herein, “scaffold” refers to a structural framework within which fungal inoculants are integrated to facilitate even distribution and effective colonization for digestion. As used herein, “pretreatment” refers to processes applied to polymer materials before introducing inoculants to enhance their susceptibility to digestion and/or degradation. As used herein, “biological additive” refers to nutrients or other compounds added to the substrate to support the growth and metabolic activity of degrading organisms. As used herein, a “bead” is a granule of unspecified shape or size. As used herein, “filler” material is non-fungal biomass material that is included in embodiments of the disclosed inoculant, including without limitation, alginate, trehalose, nutrient mixture. As used herein, a “hydrogel” is a naturally-derived or synthetic material with three-dimensional networks of hydrophilic polymers through chemical or physical cross-linking and which can absorb water. As used herein, a “natural hydrogel” includes without limitation cellulose, chitosan, collagen, alginate, agarose, hyaluronic acid, gelatin, and fibrin. As used herein, a “synthetic hydrogel” includes without limitation poly (hydroxyethyl methacrylate) PHEMA, polyethylene glycol (PEG) hydrogels, polyacrylic acid (PAA), including super absorbent polymer (SAP). As used herein, “micro-colony” or “micro-colonies” refer to isolated clusters of fungal cells. In the inventive embodiments, micro-colonies include engineered fungal cells encapsulated separately, each exhibiting distinct properties and behaviors, enabling a plurality of inoculation points, and enhanced adaptability and functionality compared to natural fungal colonies.

### Engineering Fungi to Enhance Digestion of Polymer-Based Materials Utilizing Preselected Fungi and Resulting Species Assemblies

Disclosed herein is a method for developing fungi species adapted to digest plastic materials and withstand abiotic stress. The development process involves screening potential fungi, subsequent engineering for improving digestion efficacy, and mixture of fungal inoculant with plastic waste products.

Fungi species are initially screened for inherent abilities to digest plastic materials. Selected fungi are then engineered to accept plastic as a carbon source for metabolic processes and to withstand abiotic stress conditions such as feces, urine, and other challenging environments, common in used absorbent articles and other polymer-based commercial staples.

The engineered fungi, capable of thriving under difficult conditions and utilizing plastic as a carbon source, are thereafter optimized further, including further engineering to increase the rate of plastic digestion, enabling efficient and faster digestion of plastic waste in various environments.

The engineering method and resulting engineered fungi products are highly versatile and scalable, applicable to a wide range of polymer types common to absorbent articles and other polymer-based materials.

In addition to degrading plastics, the disclosed embodiments also include methods for improving the digestion of non-plastics, cellulose, and human excrement. The immobilized fungi can be combined with other technologies to eliminate environmental toxins, reduce methane production, and sequester carbon.

The disclosed embodiments of degrading plastics using fungi improves over known methods that incorporate enzymes into products because they utilize a living organism at a relatively low inoculation rate, which are engineered to continue to grow within the presence of abiotic stressors and other fungi species using the polymer as a source of carbon. Known methods and techniques typically rely on the production of enzymes in a lab or under controlled conditions, which can be time-consuming, costly, and are not usable in common commercial settings.

In contrast, the disclosed embodiments utilize the natural ability of fungi to produce enzymes and digest plastic materials. The fungi are able to grow and spread rapidly, even at low inoculation rates, which allows use in large-scale applications. Unlike lab-produced enzymes, fungi are capable of adapting to changes in environmental conditions and can continue to grow and produce enzymes over an extended time period.

Examples shown and described below demonstrate the efficacy of the disclosed embodiments of engineered fungi to digest polymers found in diapers faster than fungi that have not been engineered according to the disclosed embodiments. Based on experimental studies, the estimated natural degradation times (without use of the disclosed embodiments) for common diaper materials are noted above.

The disclosed embodiments focus to the digestion of absorbent products, such as diapers, and the various compositions of materials used in their construction. Specifically, the embodiments show the ability of fungal species/strains or a combination thereof to target and digest the various types of polymers commonly found in absorbent products, including films, elastics, nonwovens, hot melts, and superabsorbent materials. Selecting and engineering fungal strains to specifically target and digest these polymers even in the presence of abiotic stressors, as disclosed herein, yields a highly efficient and effective product and technique for improving breaking down of absorbent products.

The ability of the fungal strains to target and digest the various types of polymers in the presence of human excrement and other abiotic stressors found in absorbent products is applicable to other types of absorbent products, including, but not limited to, adult diapers, feminine hygiene products, and other absorbent products designed for human or animal use. The fungal strains can be engineered to specifically target and digest the polymers used in each type of absorbent product, allowing for a highly effective method of digestion and disposal.

The ability of the fungal species/strains or a combination thereof to digest absorbent products also extends to other categories of products, such as plastic bags, packaging, textiles, and other common polymer-based materials. As shown herein, the disclosed methods and compositions can be adapted to target and digest the polymers found in these materials, allowing for a highly effective and versatile method of digestion and disposal.

The disclosed method and products provide significant advancement over existing technologies in the field of biodegradable absorbent products. Unlike some published studies, the disclosed embodiments are operable outside of a laboratory setting, enabling a consumer-scale commercial application. Additionally, the disclosed embodiments have achieved digestion of materials such as Super Absorbent Polymers (SAP). Still further, the disclosed embodiments do not require pre-treatment of the materials, making it a more practical and efficient solution for plastic waste management. Additional advantages of the disclosed embodiments include:
Use of the engineered fungal inoculant provides a scalable solution for degrading polymer components in absorbent products without modifying any of the plastic materials.
Composition(s) of fungal species engineered to digest long-chain carbons in plastics provide(s) for effective digestion of a wide variety of polymer types.
The disclosed embodiments are effective on a broad spectrum of plastic waste types in their commercially existing forms (i.e., without a step of pre-treating the plastic).
The disclosed embodiments are engineered to target and digest the plastic components within absorbent products, unlike existing studies that have shown fungi to grow on natural polymer components of diapers (such as cellulose, for example), but actually digest the plastic found therein.

Also disclosed herein is a method for scaling up a preselected fungus via mycelial expansion through bioprocessing, including biofermentation, to yield large scale deliverable fungi that enhance digestion of recalcitrant long chain carbon materials.

Either a clone or direct descendant seed inoculant of an engineered saprophytic species is provided. This seed inoculant is combined with a nutrient mixture tailored to the specific metabolic needs of the selected fungi species. A bioreactor facilitates maximum mycelial expansion. Once maximum expansion has been achieved, the resulting fungal biomass is homogenized after undergoing a filtration process, if needed. The homogenized hyphae are then combined with a polymer solution, such as alginate, for further processing. The processes of encapsulation and immobilization are applied subsequently to ensure the stability of the fungal inoculant. The processing techniques used for engineered saprophytic fungi allow translation of enhanced digestion and/or degradation methodologies from a lab-based concept to a commercially viable, scalable operation.

The disclosed embodiments relate to digestion of absorbent products, such as diapers, and the various compositions of materials used in their construction. Specifically, the embodiments show an ability of engineered fungal species/strains or a combination thereof to target and digest the various types of polymers found in absorbent products, including films, elastics, nonwovens, hot melts, and superabsorbent materials.

Films are commonly used in absorbent products as a layer to prevent liquid from leaking out of the product. These films are typically composed of polyethylene (PE), polypropylene (PP), or other suitable polymers. Elastics are used in absorbent products to provide stretch and fit, and are typically composed of synthetic rubber or other suitable materials. Nonwovens are used as a topsheet or acquisition layer in absorbent products, and can be composed of various polymers such as PE, PP, or bicomponent (bico) fibers. Hot melts are used as adhesives in absorbent products, and are typically composed of polymers such as polyethylene or polypropylene. Superabsorbent materials are a key component of absorbent products, and are typically composed of crosslinked polymers such as sodium polyacrylate.

By selecting and then engineering fungal strains to specifically target and digest these polymers, highly efficient and effective digestion of absorbent products can be achieved.

Importantly, the ability of the fungal strains to target and digest the various types of polymers in the presence of human excrement and other abiotic stressors found in absorbent products translates to a wide range of various absorbent products. This includes, but is not limited to, adult diapers, feminine hygiene products, and other absorbent products designed for human or animal use. The fungal strains can be engineered to specifically target and digest the polymers used in each type of absorbent product, allowing for a highly effective method of digestion and disposal.

The ability of fungal species/strains or a combination thereof to digest absorbent products also extends to other adjacent categories, such as plastic bags, packaging, textiles, and other polymer-based materials. The methods and compositions of the present invention can be adapted to target and digest the polymers found in these materials, allowing for a highly effective and versatile method of digestion and disposal.

Additionally, Examples show viability of the fungal inoculant to be immobilized and then rehydrated while maintaining effectiveness. A scaled up fungal inoculant was prepared using 2% medium viscosity sodium alginate and homogenized mycelium, with alginate percentages ranging from 1-4% (low and medium viscosity). This mixture was used in Examples 1-3 below.

### Example 1: Identification and Screening of Fungal Species to Digest Diaper Materials

Embodiments comprise utilization of fungal species, each of which have been identified and investigated for efficacy. These fungi exhibit saprotrophic abilities suitable for advancing sustainable biological processes. Fungi play a crucial role in natural ecosystems by decomposing organic matter, including the complex polymers found in wood such as lignin, cellulose, and hemicellulose. The disclosed embodiments leverage the unique saprotrophic abilities of specific fungal species, further identified as: White Rotters, Brown Rotters, and Soft Rotters (herein referred to as Species W, Species B, and Species S, respectively). The use of these uniquely capable fungal species, harnessed in unique ways, forms the basis of the inventive embodiments disclosed herein, and fungal species are frequently described herein with reference to these species categories.

White Rot Fungi (Species W): These fungi are characterized by their high production of oxidative enzymes, which allow them to break down lignin, a complex aromatic polymer in wood. They possess strong hyphae and secrete hydrophobin metabolites, which aid in their colonization and stability. These species exhibit moderate emergence lag times but grow and colonize rapidly. Taxonomically, they belong to the Basidiomycota phylum, primarily within the Agaricomycetes class and include orders such as Agaricales, Hymenochaetales, and Polyporales. Specific species utilized include Pleurotus ostreatus, Pleurotus djamor, Dichomitus squalens, Trametes versicolor, Phanerochaete chrysosporium, Lentinula edodes, and Phellinus pini.

Brown Rot Fungi (Species B): Brown rot fungi are notable for their high production of hydrolytic enzymes, which efficiently digest cellulose and hemicellulose in wood, leaving behind a brown, crumbly residue of lignin. These fungi are adept at outcompeting microbial contaminants and typically have a high emergence lag time but exhibit moderate growth and colonization rates once established. They are also within the Basidiomycota phylum and the Agaricomycetes class, with representatives in orders like Gloeophyllales and Polyporales. Species utilized include Inonotus obliquus and Fomitopsis spraguei.

Soft Rot Fungi (Species S): Soft rot fungi thrive in harsher conditions where other fungi may not survive, such as in wood with high moisture content or in environments with extreme temperatures or PH levels. These fungi show high compatibility with other fungal species, making them useful in diverse microbial communities. They exhibit moderate to fast emergence lag times but grow and colonize slowly. Taxonomically, they belong to the Ascomycota phylum, primarily within the Dothideomycetes and Sordariomycetes classes, with orders including Hypocreales, Xylariales, and Eurotiales. Specific species used include Aureobasidium pullulans, Pestalotiopsis microspora, Aspergillus sp., Fusarium sp., Aspergillus versicolor, and Aspergillus fumigatus.

TABLE 1
Taxonomic and functional classification of species

| | | |
|---|---|---|
| Phylum | Class | Order |

Species S

| | | |
|---|---|---|
| Ascomycota | Dothideomycetes | |
| Ascomycota | Sordariomycetes | Hypocreales |
| Ascomycota | Sordariomycetes | Xylariales |
| Ascomycota | Eurotiomycetes | Eurotiales |

Species W

| | | |
|---|---|---|
| Basidiomycota | Agaricomycetes | Agaricales |
| Basidiomycota | Agaricomycetes | Hymenochaetales |
| Basidiomycota | Agaricomycetes | Polyporales |

Species B

| | | |
|---|---|---|
| Basidiomycota | Agaricomycetes | Gloeophyllales |
| Basidiomycota | Agaricomycetes | Polyporales |

TABLE 2
Preselected Fungal Species Examples

| | | | |
|---|---|---|---|
| Species | Class | Order | Phylum |

Species S

| | | | |
|---|---|---|---|
| Aureobasidium pullulans | Dothideomycetes | Dothideales | Ascomycota |
| Pestalotiopsis microspora | Dothideomycetes | Xylariales | Ascomycota |
| Aspergillus sp. | Eurotiomycetes | Eurotiales | Ascomycota |
| Fusarium sp. | Sordariomycetes | Hypocreales | Ascomycota |
| Aspergillus versicolor | Eurotiomycetes | Eurotiales | Ascomycota |
| Aspergillus fumigatus | Eurotiomycetes | Eurotiales | Ascomycota |

Species W

| | | | |
|---|---|---|---|
| Pleurotus ostreatus | Agaricomycetes | Agaricales | Basidiomycota |
| Pleurotus djamor | Agaricomycetes | Agaricales | Basidiomycota |
| Dichomitus squalens | Agaricomycetes | Polyporales | Basidiomycota |
| Trametes versicolor | Agaricomycetes | Polyporales | Basidiomycota |
| Phanerochaete chrysosporium | Agaricomycetes | Polyporales | Basidiomycota |
| Lentinula edodes | Agaricomycetes | Agaricales | Basidiomycota |
| Phellinus pini | Agaricomycetes | Hymenochaetales | Basidiomycota |

Species B

| | | | |
|---|---|---|---|
| Inonotus obliquus | Agaricomycetes | Hymenochaetales | Basidiomycota |
| Fomitopsis spraguei | Agaricomycetes | Polyporales | Basidiomycota |

Fungal Digestion of Polymers refers to the extracellular process by which fungi break down complex polymeric materials. This process involves several steps: the fungi first sense and recognize the polymer as a food source, then colonize and surround it with a biofilm. Within this biofilm, the fungi excrete extracellular enzymes, such as oxidative and hydrolytic enzymes, which initiate the breakdown of the polymer into intermediate compounds. These intermediates are then assimilated by the fungi. Thus, as used herein, the term “digest” encompasses the entire sequence of recognizing, colonizing, enzymatically degrading, and assimilating the polymer material.

TABLE 3
Fungal Digestion of Polymers

| | | | | Biodete- | Biofrag- | Bioassim- | Mineral- |
|---|---|---|---|---|---|---|---|
| Phase | Recognition | Colonization | Secretion | rioration | mentation | ilation | ization |
| Description | Recognize | Grow and | Secrete | Weaken polymer | Hydrolytic | Intermediates, | Hydrolysis |
| | and sense | surround | biofilm to | mechanically | enzymes | such as | products |
| | polymer as a | food source. | create | to expose | cleave carbon | oligomers, | transfer within |
| | food source. | | environment | surface area | chains and | are taken | the cell wall. |
| | | | for | and utilize | produce | up by fungi. | |
| | | | degradation. | oxidative | intermediates. | | |
| | | | | enzymes to | | | |
| | | | | create | | | |
| | | | | intermediates. | | | |
| Measures | Fungi grow | Fungal | Enzyme | Weakened | Lower | Sustained | Sustained |
| | towards | colonization | production. | material, | molecular | metabolism | growth on |
| | polymer. | of polymer. | | oxidation of | weight, | on substrate. | substrate. |
| | | | | chemical | crystallinity, | | |
| | | | | structure. | polymer | | |
| | | | | | backbone, and | | |
| | | | | | hydrolysis | | |
| | | | | | product | | |
| | | | | | detection. | | |
| Methods | Inoculate at | Visible | SEM, | FTIR −> | Chemical | Sustained | Sustained |
| | a distance | colonization. | Colorimetric | Oxidation. | structure | activity of | activity and |
| | from polymer | | Assays. | | from FTIR. | previous | further growth |
| | and observe | | | | Thermal | methods, | visually |
| | growth towards | | | | stability | with | indicated with |
| | polymer on | | | | from TGA, | inoculation | inoculation |
| | substrate. | | | | DSC. | ratio <1:1. | rate <1:1. |

The phases of the digestion process and associated engineering processes and procedures utilized in engineering the disclosed embodiments of engineered fungi are described in detail below:

Recognition—Fungi recognize the polymer as a potential food source through chemical signals or structural components unique to the polymer. The engineering process disclosed herein includes using previously digested polymers, prompting fungi to extend hyphae toward the polymer rather than ignoring it. To determine if the recognition phase is occurring, fungal growth towards the polymer can be observed by inoculating nutrient media petri dishes with the fungi and placing small pieces of the polymer at a distance from the inoculum. In full polymer product field studies, fungal colonization on the polymer surface can be monitored over time by visual inspection and microscopy to confirm directed growth towards the polymer.

Colonization—Upon recognition, fungi begin to colonize the polymer surface, attaching and growing on it. This phase is adapted to prepare for full digestion by developing fungal hyphae that establish a stable environment. To determine if colonization is occurring, visual observation and microscopy can be used to detect the attachment and growth of fungal hyphae on the polymer surface. The area of fungal growth can be calculated by measuring the extent of colonization, and growth rate can be determined by tracking the expansion of the fungal network over time.

Biofilm Secretion—Within the biofilm, fungi secrete a variety of extracellular substances, including enzymes and hydrophobins. These adaptations allow the fungi to quickly secrete the right mixture for the specific polymer sensed. Along with visual observation and microscopy, colorimetric assay kits can be used to detect the presence of enzyme production when fungi colonize plastics. These techniques confirm biofilm formation and the active involvement of the fungi in the digestion process.

Biodeterioration—Fungi weaken the polymer mechanically and through oxidative enzymes, forming carbonyl groups. This process also targets crosslinks and amorphous groups of the polymer to facilitate further breakdown, particularly for homochains resistant to immediate hydrolysis. Tensile testing can be used to identify changes in the strength and elasticity of the polymer, indicating structural weakening. Additionally, FTIR can be employed to examine changes in functional groups within the polymer structure, specifically identifying oxidation by detecting increased intensity in the O—H band compared to controls.

Biofragmentation—Hydrolytic enzymes cleave the polymer carbon chains, producing intermediate products such as oligomers. The fungi are adapted to produce the necessary enzymes to break down the polymer into intermediates suitable for assimilation. Biofragmentation can be identified through various analytical methods on the polymer. FTIR is used for homochains, where an increase in the carbonyl index indicates the cleavage of the polymer backbone. For heterochains, DSC is used to inspect thermal stability and properties like crystallinity. TGA is most appropriately used on crosslinked polymer SAP to identify mass loss of the sample during heating, indicating decomposition and fragmentation.

Bioassimilation—The intermediate products from biofragmentation, such as oligomers, are taken up by the fungal cells. These intermediates are then metabolized, providing energy and building blocks for fungal growth. Fungi have unique capabilities to initiate and catalyze digestion for biodeterioration and biofragmentation. Once these steps have been identified, the intermediate byproducts can be taken up by the fungi. This uptake is indicated and observed by sustained metabolism when polymers are inoculated with a low inoculation ratio, demonstrating that the fungi are surviving off this carbon source.

Mineralization—Hydrolysis products are transferred within the cell wall and converted into microbial biomass. The sustained lifecycle and continued growth on the polymer substrate indicate the fungi are effectively utilizing the plastic as a food source and continuing all previous processes. Mineralization is determined by sustained growth on the polymer product with a low inoculation rate, indicating that the fungi are effectively utilizing the degraded polymer components as a carbon source for continued metabolic activity and further colonization of substrate.

### Polymer Testing

The disclosed embodiments are aimed primarily, but not exclusively, to the field of fluid absorbent articles, particularly diapers and other personal waste disposal items. Additionally, embodiments of novel absorbent materials and constructions that can be used in a variety of absorbent products to provide improved absorbency, fluid retention, and overall performance while ensuring comfort, fit, and ease of use are disclosed. The methods provided herein do not require new materials to achieve a more sustainable product, thereby eliminating the trade-off between performance vectors and sustainability.

Polymers used in the herein methods and products are categorized into homochains, heterochains, crosslinked polymers, and copolymers. Homochain polymers, such as polyethylene (PE) and polypropylene (PP), are not susceptible to hydrolysis and require oxidation for digestion and degradation. These materials are tested in various diaper components including backsheet nonwoven, backsheet film, and leg cuffs. Heterochain polymers, including polyethylene terephthalate (PET) and polyurethane (PU), are more susceptible to hydrolysis but have complex structures. These are tested in the acquisition and distribution layer (ADL) and elastic strands, respectively.

Crosslinked polymers and copolymers also play a crucial role in the disclosed methods and products. The superabsorbent polymer (SAP) used is a hydrogel composed of sodium polyacrylate (NaPA) crosslinked with approximately 5% polyethylene glycol (PEG). This hydrogel is designed to enhance fluid retention capabilities. Additionally, copolymers such as acrylonitrile butadiene styrene (ABS) are utilized in adhesive applications, combining properties of different monomers to improve adhesive performance.

The materials tested herein include a variety of polymer families used in different diaper components. For instance, backsheet nonwoven materials made of polypropylene (PP) and backsheet films made of polyethylene (PE) represent homochain polymers. The acquisition and distribution layer, composed of polyethylene terephthalate (PET), and elastic strands made of polyurethane (PU) represent heterochain polymers. The SAP, a crosslinked hydrogel, and adhesives composed of ABS, a copolymer, further demonstrate the range of materials used.

The absorbent products, such as diapers, include multiple layers designed for specific functions. The topsheet is typically a nonwoven material made from PE or PP and is intended for skin contact. The acquisition layer quickly wicks liquid into the absorbent core, which is composed of cellulose fibers and SAP to capture and hold liquid. The backsheet, made from PE or PP, prevents liquid from leaking out of the diaper. Additional components include adhesives, elastics, and fasteners to ensure functionality and fit.

This versatile approach allows for the scaling and adaptation of these materials to other combinations within absorbent product layers. Furthermore, the methodologies and materials developed in this invention can be broadly applied to a wide range of plastic products, offering scalable solutions for waste treatment, not limited to absorbent products.

TABLE 4
Diaper Materials

| Polymer | | | | Material |
|---|---|---|---|---|
| Family | Diaper Material | Code | Polymer | Form |
| Homochain | Backsheet | BSNW | PP | NW |
| | Nonwoven | | | |
| | Backsheet Film | BSF | PE | Film |
| | Front Ear | FENW | PP or PE | Laminate |
| | Nonwoven | | | |
| | Landing Zone | LP | PP & PE | Laminate |
| | Leg Cuff Nonwoven | LCNW | PP & PE | Laminate |
| | Topsheet | TSNW | PP | NW |
| | Nonwoven | | | |
| Homochain - | Adhesive | AE | Copolymer | Hot Melt |
| Copolymer | | | (ABS) | |
| Heterochain | Acquisition and | ADL | PET | NW |
| | Distribution Layer | | | |
| | Elastic Strands | ES | PU | Elastic |
| Heterochain- | SAP | SAP | NaPA | Absorbent |
| Crosslinked | | | PEG | Hydrogel |

### Example 2: Diaper Materials Digestion

In Example 2, 30 species from the categories of White Rot, Brown Rot, and Soft Rot fungi were mixed in sequentially lower inoculation rates with superabsorbent polymer (SAP) starting with fungi inoculum:plastic weight ratio of 99:1; then 10:1; then 4:1; and then 1:1. From this adaptation process, 12 species were selected for their effective recognition and colonization of SAP as a food source. The adapted cultures were then applied at a 1:1 inoculation ratio to various diaper materials: Topsheet Nonwoven (PP), Backsheet Nonwoven (PP), Core Wrap (PP), Backsheet Film (PE), and SAP. The average growth rate was observed over 30 days. FIG. 1 provides a flow chart of Example 2, photographs of petri dishes showing substantial growth for Species S and Species W, and growth rate data for Species W on different plastic materials.

To confirm biofilm secretion and enzyme production, the fungal species and strains were compared to known polymer-degrading proteins for assessing ability and gene expression cultured with TS. Specific enzyme activities were qualitatively evaluated using colorimetric assays:
Laccase: The formation of a dark green halo on plates supplemented with ABTS indicates positive laccase secretion.
Lignin Peroxidase: The formation of a clear halo on plates supplemented with azure B indicates positive lignin peroxidase secretion.
Manganese Peroxidase: The formation of a reddish-brown halo on plates supplemented with guaiacol indicates positive manganese peroxidase secretion.

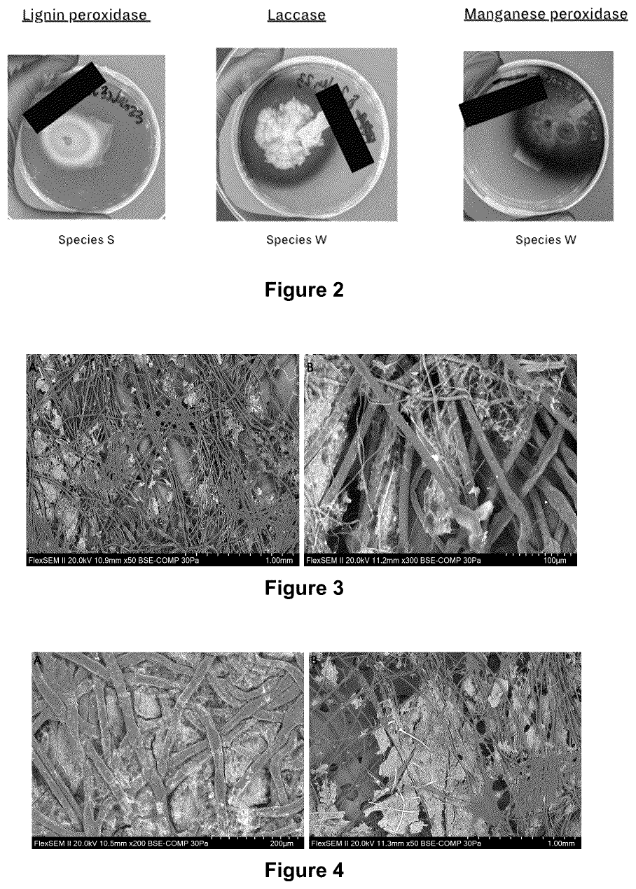

Qualitative analyses showed diverse and abundant enzyme production in the presence of plastics from the adapted fungal species. FIG. 2 depicts colorimetric analysis demonstrating lignin peroxidase from Species S, and laccase and manganese peroxidase [C] production from Species W.

### Example 3: Inoculum Optimization for Diaper Material Digestion

In Example 3, a 1:1 inoculum-to-plastic weight ratio was employed, i.e., weight of fungal biomass and nutrient compound equal to weight of the plastic waste being treated. This ratio is significant as it confirms that the fungi have sufficient resources to efficiently colonize and digest the plastic materials. In preferred embodiments of the inoculum described in greater detail below, the inoculum is effective at inoculating plastic material at a weight ratio below 1:1 inoculum:plastic, as shown representatively in FIG. 1B.

The wet inoculum utilized in Example 3 comprises 4% fungal biomass and 96% crosslinked sodium alginate. The fungal biomass were cultured from three screened and adapted fungal species of Species S and Species W (two Species S and one Species W). This inoculum was applied to each individual layer of the diaper which were tested for 30 days on the following materials: Backsheet Nonwoven, Backsheet Film, Front Ear Nonwoven, Landing Zone, Leg Cuff Nonwoven, Topsheet Nonwoven, Adhesive, Acquisition and Distribution Layer, Elastic Strands, and SAP. An environmental control sample was run along with samples of the fungal treatments, and results were compared from these treatments and raw (bulk) undegraded/undigested materials. The digestion results for each material layer were analyzed, encompassing both biological activity and polymer characterization. The data shows that these enzymes produced by the adapted focal fungal strains digest polymers from families of homochains, heterochains, and crosslinked polymers. The data additionally shows changes in the physical and chemical structure (oxidation), and cleavage of polymer bonds (hydrolysis) from fungal treatments when compared to virgin bulk material and environmental controls, indicating successful recognition, colonization, secretion, biodeterioration, and biofragmentation by the adapted fungal samples.

Additionally, presence of mycelial growth and enzymatic activity on the surfaces of the respective diaper materials provides evidence of digestion. All of the diaper materials tested were successfully recognized and colonized by one or more of the adapted species. FIG. 3 are SEM images showing that TS was effectively inoculated with Species S (thinner threads throughout the matrix are fungal hyphae). FIG. 4 are SEM images showing that BSNW was effectively inoculated with Species W (digestion of the polypropylene non-woven material can be seen).

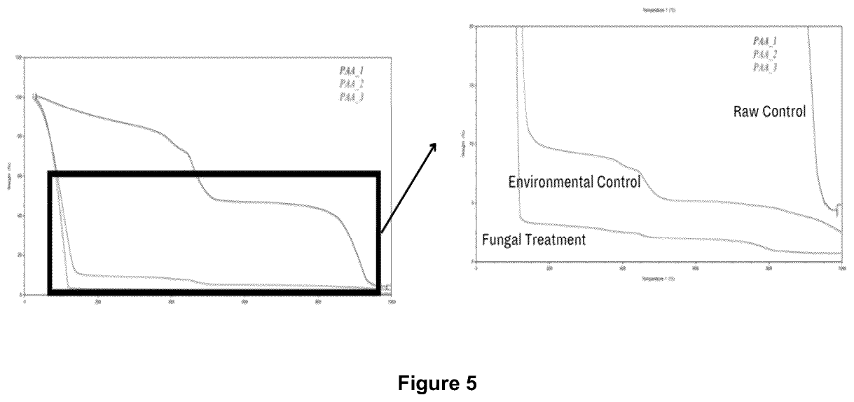

Thermogravimetric analysis (TGA) reveals that the Fungal Treatment sample with Species W of SAP (NaPA Crosslinked with PEG) is less thermally stable throughout all phases of tests compared to the Raw SAP Control and Environmental Control samples. As shown in FIG. 5, the Fungal Treatment sample exhibited a significant initial weight loss of approximately 95-96% around 100° C., indicating high hydrophilicity, likely due to increased porosity or surface area from fungal digestion of the crosslinking structures. Further weight loss occurred between 100° C. and 500° C., indicating decomposition of the polymer bonds, with smaller, successive weight losses suggesting that shorter polymer chains, a result of fungal digestion, digest at lower temperatures. In the high-temperature range (500° C.-1000° C.), the Fungal Treatment sample left behind about 2.08% residue, the smallest amount among the samples, indicating thorough breakdown of the organic structure. These results clearly demonstrate that the Fungal Treatment sample is less thermally stable across all temperature ranges, highlighting the significant impact of fungal digestion on SAP.

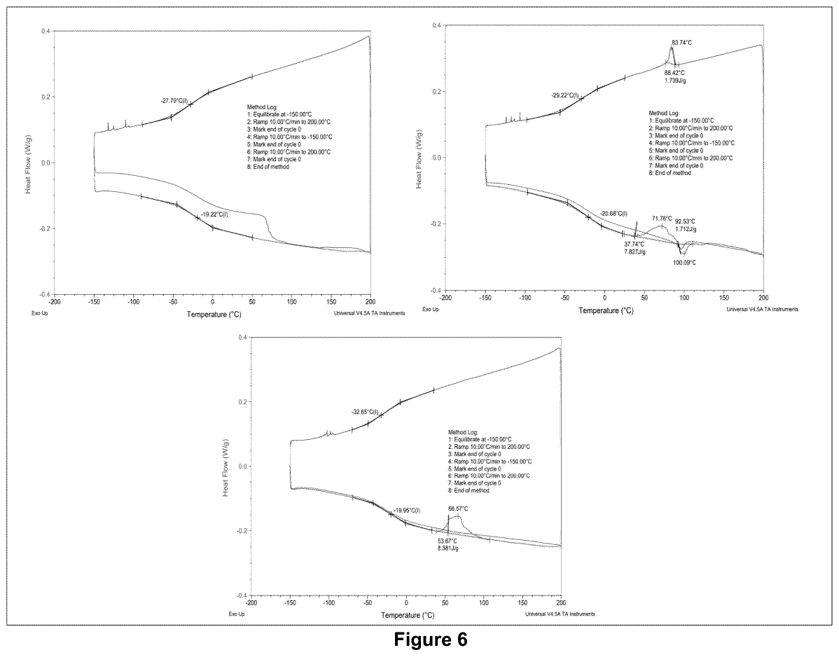

Additionally, differential scanning calorimetry (DSC) results indicate significant digestion in hotmelt adhesives subjected to environmental exposure and fungal treatment with Species W. As shown in FIG. 6, both the Environmental Control and Fungal Treated samples show lower glass transition temperatures (Tg) compared to the raw adhesive, suggesting digestion due to molecular weight reduction or structural changes. The Environmental Control sample shows crystallization behavior with an onset temperature of 37.74° C. and a peak temperature of 71.76° C. during the first heating cycle, whereas the fungal treatment significantly alters this behavior, indicating structural changes. Additionally, the Environmental Control sample exhibits reduced crystalline content upon the second heating cycle, with an onset temperature of 92.53° C., a peak temperature of 100.09° C., and specific energy of 1.712 J/g, while the Fungal Treated sample lacks a melting peak, indicating extensive digestion and the elimination of crystalline regions. These results collectively suggest that both environmental exposure and fungal treatment digest the adhesive, with fungal treatment causing more significant structural changes and a complete loss of crystallinity.

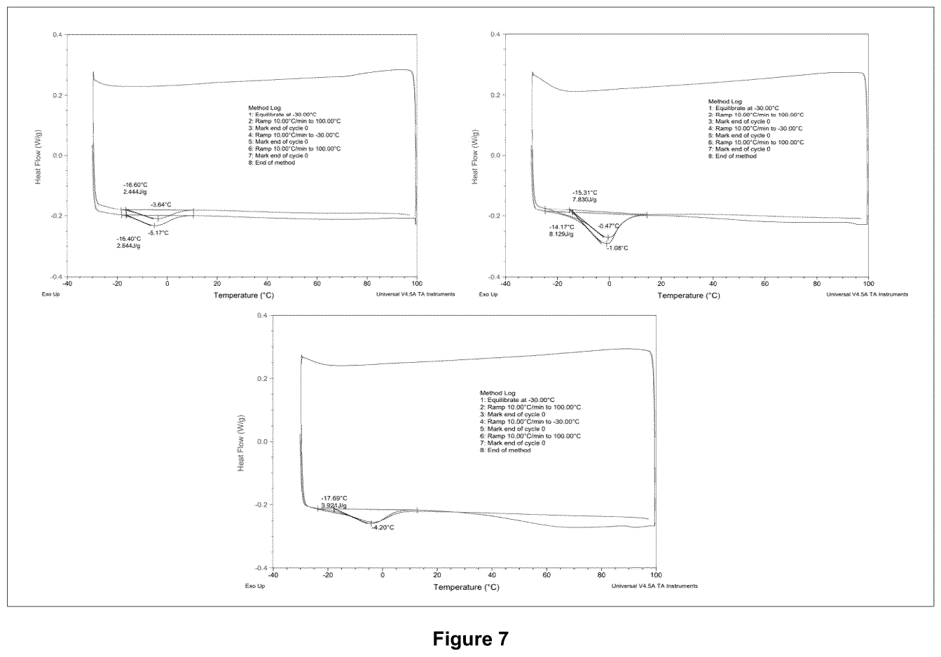

Likewise, as shown in FIG. 7, DSC results for elastics highlight significant digestion in the Fungal Treated (Species W) samples compared to the Raw and Environmental Control samples. While raw elastics exhibit minimal digestion between cycles, indicating a stable structure, the Environmental Control samples show increased crystallinity with higher specific energy (8.129 J/g during the first heating cycle), suggesting environmental conditions enhance the polymer's structure with slight digestion between cycles. In contrast, the Fungal Treated samples demonstrate moderate crystallinity (specific energy of 3.921 J/g during the first heating cycle) and significant digestion compared to the raw and environmental controls. The stable structure between heating cycles in the fungal-treated samples suggests that initial fungal treatment caused substantial structural changes, leading to pronounced digestion and moderate crystallinity compared to the other samples.

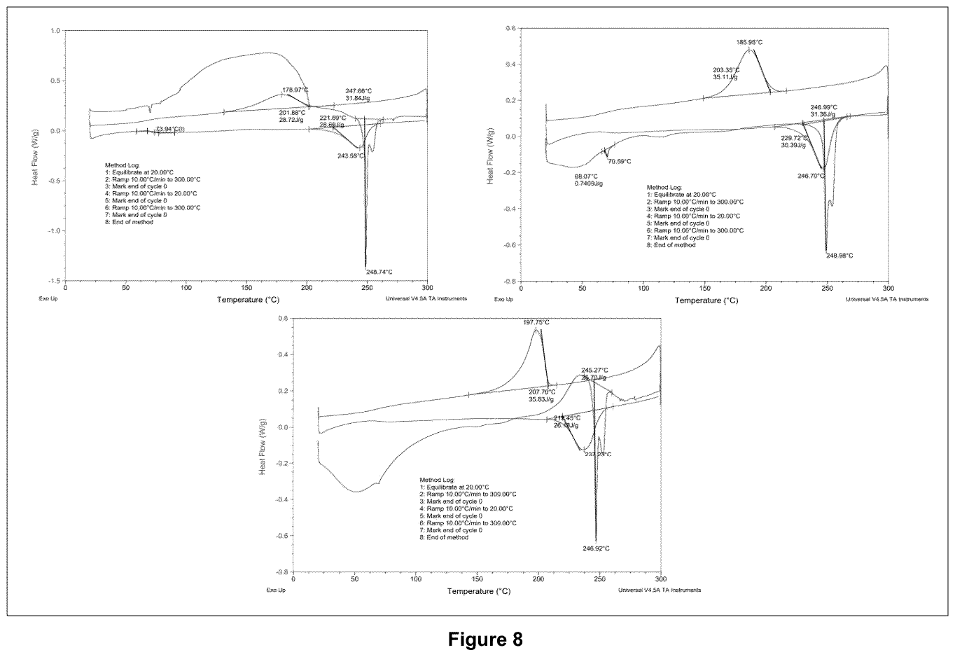

FIG. 8 shows DSC results for PET nonwoven layers, revealing significant digestion in the fungal treated samples (Species S) compared to the raw and environmental control samples. The environmental control samples show minor digestion with slightly reduced specific energy during the endothermic peak (melting), indicating minor crystalline content reduction. In contrast, the fungal treated samples maintain high specific energy during the endothermic peak, indicating less impact on crystallinity but exhibit a secondary melting peak, suggesting complex thermal behavior and potential formation of different crystalline phases or structures due to fungal treatment. These findings indicate that while the raw sample shows stable crystallinity and thermal properties, the fungal treated sample exhibits significant structural changes and more complex thermal behavior.

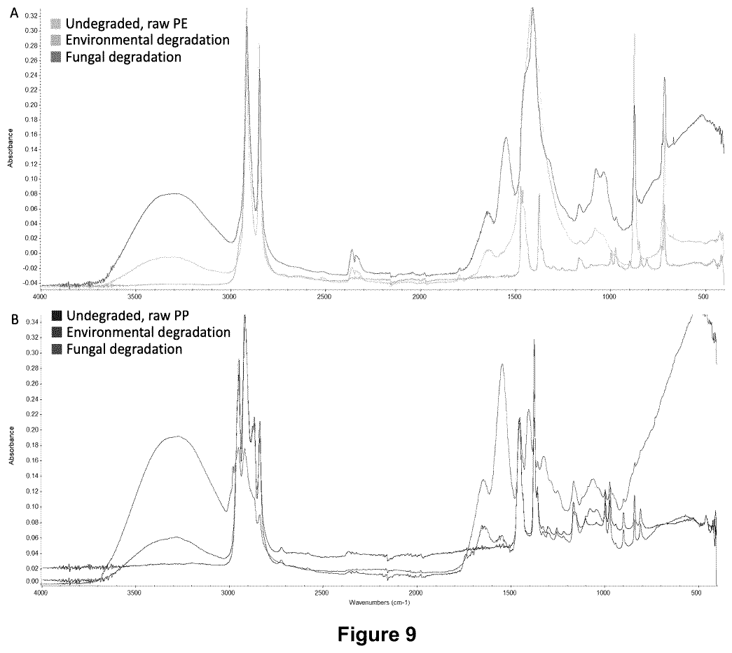

Fourier transform infrared spectroscopy (FTIR) was used to analyze the diaper materials before and after inoculation with Species W with results shown in FIG. 9. The FTIR results indicate a reduction in the carbon backbone and oxidation of the materials after exposure to the fungi providing additional evidence of digestion that is more than that caused by environmental factors. See FIG. 9, wherein plot A shows polyethylene and plot B shows polypropylene for the respective undigested sample, environmentally digested sample, and fungal digested sample. Environmental digestion was due to UV and heat. In both polypropylene and polyethylene, sharp peaks at 2900 cm⁻¹ correspond to C—H stretching vibration of methyl groups and represent the long carbon backbone. Additionally peaks at 1460 cm⁻¹ and 1375 cm⁻¹ represent CH₂ and CH₃ accessory groups on polypropylene. Oxidation evidence can be seen by reduction in these peaks, as well as new peaks forming at 1740 cm⁻¹ (carbonyl groups), 1100 cm⁻¹ (ether and alcohol groups) and 3350 cm⁻¹ (alcohols).

### Example 4: Adaptation of Fungi to Polymers

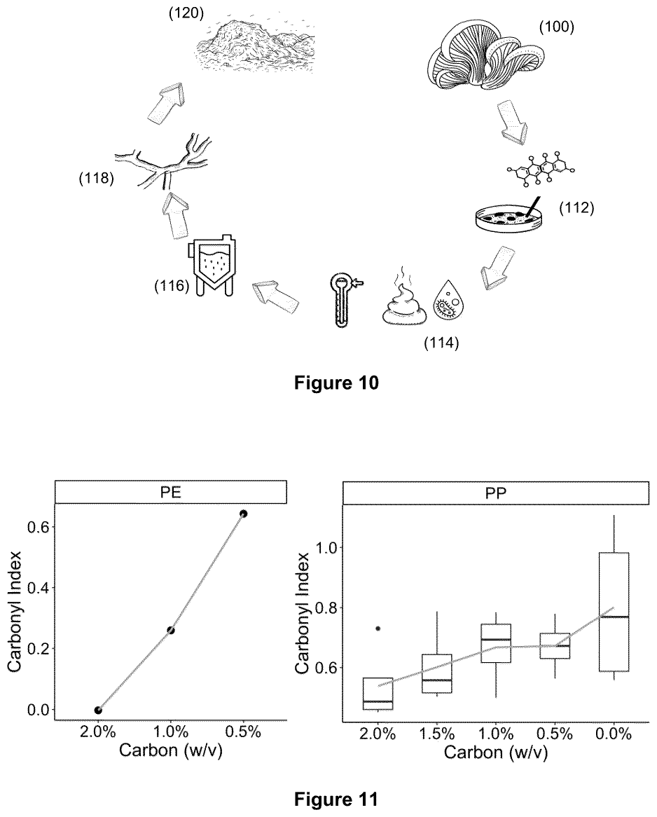

An exemplary adaptation process is shown generally in FIG. 10 depicting generally a raw fungal species 100 that is engineered via adaptation to plastics 112, followed by further adaptation to abiotic stresses 114. The biomass of the adapted fungal species is thereafter recovered from the agar petri dish and used to form an inoculant. In Example 4, Species S was adapted to digest polyethylene (PE), and Species W was adapted to digest polypropylene (PP). In engineering this adaptation, the fungi were gradually conditioned to use these complex polymers as their primary carbon source by systematically reducing access to simpler carbon sources such as glucose and sucrose.

The adaptation process began by preparing a mixture of the selected polymer (Species S with PE and Species W with PP) and a carbon nutrient food source. Initially, the fungi utilized both the polymer and the additional food source for growth and metabolism. Over successive generations, the concentration of the additional carbon nutrient was gradually reduced. This forced the fungi to rely more on the polymer as their primary carbon source, thereby increasing their enzymatic expression needed to digest the polymers. This systematic process yields an engineered fungal species that grows considerably more rapidly using plastic carbon than its naturally occurring counterpart. Fungal species can be further engineered to be more accepting of certain type of polymer materials as carbon source for metabolism via a similar sequential process.

Each generation was cultured for 2 weeks prior to transferring to the next culture (with decreased carbon concentration in the culture media). The contents of the generations in Example 4 are identified below.
Generation 1 (100% carbon): 5 g glucose, 5 g malt extract, 2.5 g yeast extract, 0.5 g MgSO₄*7H₂O, 1 g KH₂PO₄ in 500 mL ultrapure water.
Generation 2 (75% carbon): 3.75 g glucose, 3.75 g malt extract, 1.875 g yeast extract, 0.5 g MgSO₄*7H₂O, 1 g KH₂PO₄ in 500 mL ultrapure water.
Generation 3 (50% carbon): 2.5 g glucose, 2.5 g malt extract, 1.25 g yeast extract, 0.5 g MgSO₄*7H₂O, 1 g KH₂PO₄ in 500 mL ultrapure water.
Generation 4 (25% carbon): 1.25 g glucose, 1.25 g malt extract, 0.625 g yeast extract, 0.5 g MgSO₄*7H₂O, 1 g KH₂PO₄ in 500 mL ultrapure water.
Generation 4.1 (15% carbon): 0.75 g glucose, 0.75 g malt extract, 0.375 g yeast extract, 0.5 g MgSO₄*7H₂O, 1 g KH₂PO₄ in 500 mL ultrapure water.
Generation 4.2 (10% carbon): 0.5 g glucose, 0.5 g malt extract, 0.25 g yeast extract, 0.5 g MgSO₄*7H₂O, 1 g KH₂PO₄ in 500 mL ultrapure water.
Generation 4.3:0.5 g glucose, 0.5 g yeast extract in 500 mL ultrapure water.
Generation 5 (0% carbon): 0.0 g glucose, 0.0 g malt extract, 0.0 g yeast extract, 0.5 g MgSO₄*7H₂O, 1 g KH₂PO₄ in 500 mL ultrapure water.

Fungal strains were cultured with the respective plastic polymer segments for 2 weeks in each Generation prior to being transferred to the next sequential culture Generation, first by removing plastic polymers, scraping all fungal mycelium off, and drying the mycelium in a dehydrator at 95° C. for 12 hours. FTIR data collected during this process indicated an improvement in the carbonyl index, a marker of polymer digestion for both PE (Species S) and PP (Species W), as shown in FIG. 11. The FTIR results showed that, as the simpler carbon sources were reduced, there was a significant increase in digestion activity of the respective adapted fungi species, demonstrating the fungi's enhanced capability to break down the complex polymers.

### Example 5: Field Testing

The efficacy of the engineering/adaptation was verified through field trials (Example 5). In these trials, a 0.3 g inoculum of fungi identified as Species S₁ and Species W₁ was mixed with a 30 g polymer product. Both adapted and non-adapted inoculum conditions were tested. The mixtures were left in an open-air environment and recovered after six weeks. Backsheet Film (BSF), made of polyethylene (PE), and backsheet nonwoven material, made of polypropylene (PP), were recovered and tested.

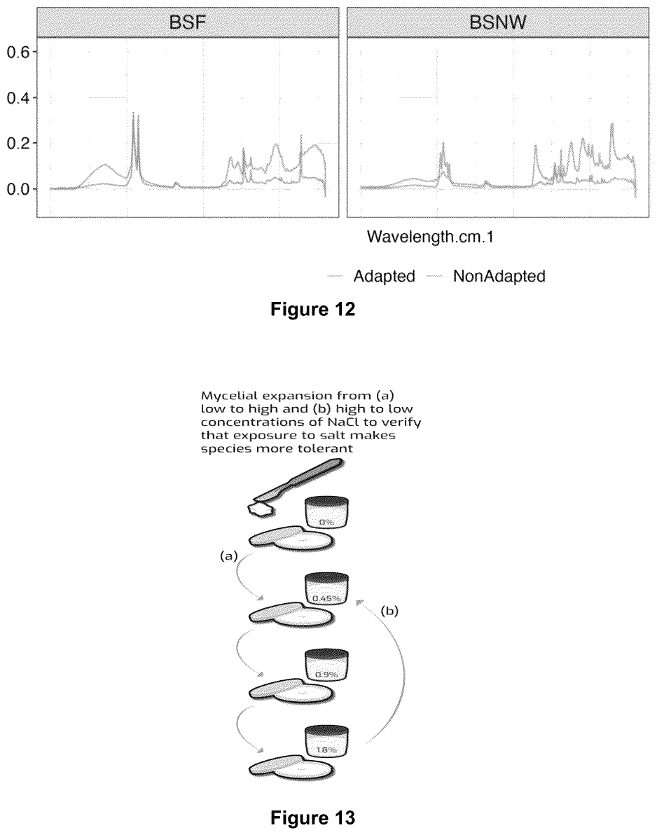

The FTIR plot of FIG. 12 shows significant differences between the adapted and non-adapted Species S₁ and W₁, respectively. The adapted inoculum exhibited increased oxidation peaks and more significant changes in polymer structure. The adapted Species S₁ and W₁ successfully digested polyethylene and polypropylene, respectively, highlighting the scalability to real world disposal environments from fungi maintaining enzymatic expression outside of lab conditions.

### Example 6: Adaptation to Contaminants

Example 6 focuses on adaptation of fungi to abiotic stressors such as urine, enhancing their performance on substrates like plastics, urea, and feces. This example demonstrates the adaptation of fungi to saline conditions, focusing on growth, development, and enzymatic activity under different salt concentrations. A graphical representation of an abiotic stress adaptation process like that used in Example 6 is shown as FIG. 13. Species W adapted as per Example 4 above was cultured on Potato Dextrose Agar (PDA) and liquid media. The fungi were progressively exposed to 0.45%, 0.9%, and 1.8% salt concentrations in liquid cultures, with positive controls (no salt) and negative controls (no inoculum). The cultures were incubated at 30° C. and monitored every 3 days for 43 days, measuring biomass and enzyme activity. Every 3 days, 15 ml of liquid was extracted from each jar for analysis. Parameters such as pH, conductivity, salinity, oxidation-reduction potential (ORP-MV), and total dissolved solids (TDS) were measured from the liquid culture to monitor the environmental conditions and the fungi's metabolic activity. These parameters indicated sustained fungal growth without dying. The fungi adapted well to increased salinity, demonstrating significant growth and enzymatic activity at highest saline concentrations. The engineered fungal species adapted to plastics and abiotic stresses form the basis of the disclosed embodiments, and were used in the Examples that follow.

### Example 7: Combined Soiled Product and Inoculum Field Test

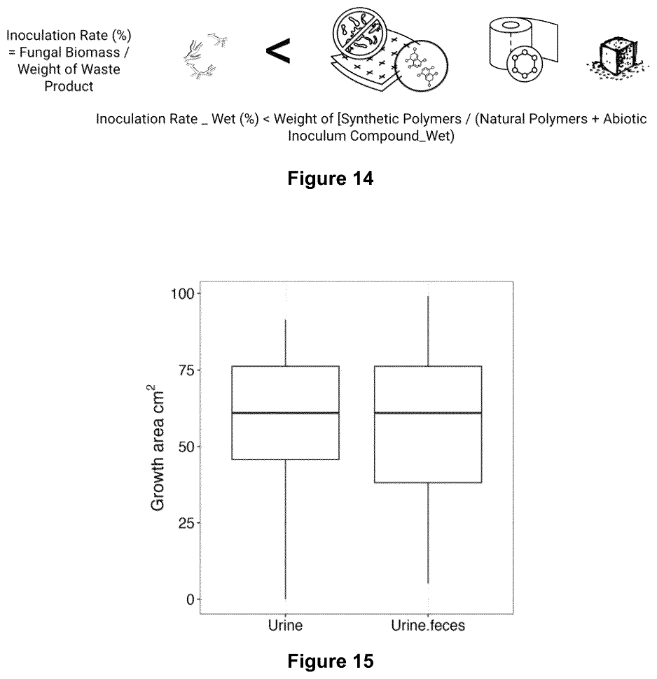

As graphically depicted in FIG. 14, the disclosed embodiments of the inoculum with engineered fungal species are effective at inoculating plastic waste in “field” conditions with other waste including natural polymer waste at an inoculation rate less than 1:1 fungal inoculant to waste. Example 7 shows that the inoculum is effective in conditions with fully soiled products and the fungal inoculum left in an open field environment. 650 soiled diapers of the same composition were collected from daycares. The diapers were inoculated with fungal inoculum at varying percentages. The contamination type, whether urine or feces, was recorded. Nine engineered species of each of Species W, Species S and Species B were tested, with three replicates of each condition placed together in a bucket. The wet inoculum utilized in Example 7 comprised 4% fungal biomass and 96% crosslinked sodium alginate. The fungal biomass was cultured from screened and adapted fungal species per the above Examples, scaled up using liquid fermentation. The mycelial biomass was recovered, homogeneously mixed with 2% sodium alginate, and extruded into a calcium chloride solution for crosslinking, resulting in beads approximately 3 mm in diameter. Notably, the inoculum ratio recorded includes only the weight of the dry diaper, excluding weight of the contaminants.

Samples were collected for FTIR analysis after 1 month, and each sample was observed and collected after 3 months for further analysis. FIG. 15 shows the total growth area on the diaper with box and whiskers plots illustrating the contamination types (urine, and urine and feces) for all species, demonstrating that the engineered fungi in the inoculant were able to outcompete biotic and abiotic contaminants to colonize.

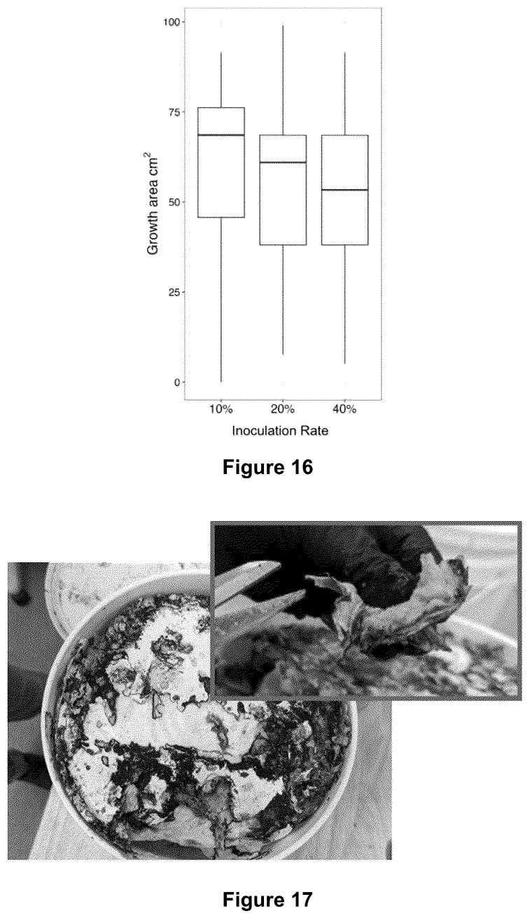

FIG. 16 shows the total growth area for inoculation rates of 40%, 20%, and 10% (inoculation rate as defined in FIG. 14), indicating that the embodiment of fungal inoculant effectively colonized at even low inoculation rates.

After one month, significant fungal growth was observed, and after two months, a Species W sample had grown completely through the buckets of diapers, as shown in the photographs of FIG. 17 for Species W.

Carbonyl index (intensity or area change of carbonyl group in FTIR spectra) was used as a quantitative data point to measure the level of plastics digestion (as a proxy for general digestion). The presence of carbonyl groups indicates that the polymer has been digested and facilitates further digestion due to the instability of carbonyl groups. A carbonyl index can be calculated from a FTIR spectrum by analyzing the peaks that indicate the presence of carbonyl and methylene groups (see Formula 1 below). As oxidation occurs during digestion, methylene groups will be reduced eventually transforming into carbonyl groups. By comparing the carbonyl index (CI) of a digested sample with an undigested control, the amount of digestion can be assessed.

$$CI = \frac{\begin{matrix}Area of absorption peak of carbonyl \\ bond ( 1850 - 1650 cm - 1 )\end{matrix}}{\begin{matrix}Area of absorption peak for methylene \\ bond ( 1500 - 1420 cm - 1 )\end{matrix}} \qquad ( Formula 1 )$$

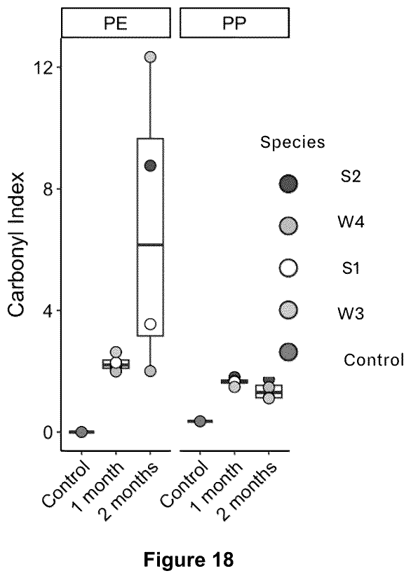

FTIR data established that all species showed signs of polymer digestion after one month, with calculations of the carbonyl index (CI>1) indicating good digestion compared to the controls. After two months, the carbonyl index increased significantly to an average of 6, while PP still maintained a CI>1 but did not see as much of an increase. As shown in FIG. 18, all engineered species (Species S, Species W) had a higher digestion efficiency than the respective non-engineered control.

After two months, a sample was also analyzed via Differential Scanning calorimetry (DSC). This sample was a composite from Backsheet Film (PE) and Backsheet Nonwoven (PP), and one heating cycle was utilized for this method to calculate crystallinity. Two Species W samples showed significant decreases in crystallinity, indicating substantial digestion. For PE, Species W3 exhibited 79.96% crystallinity and Species W4 exhibited 69.08% crystallinity. For PP, Species W3 showed 31.59% crystallinity and Species W4 showed 92.25% crystallinity.

TABLE 5
Crystallinity reduction from fungal Species W samples
Crystallinity Reduction From DSC

| | PE | PP |
|---|---|---|
| Species W3 | 79.96% | 31.59% |
| Species W4 | 69.08% | 92.25% |

Also disclosed herein are embodiments of a composite fungal consortia configured for enhanced digestion of polymer waste products. The preselected fungi are not naturally occurring in the specific combinations, allowing for the adaptation to both biotic and abiotic factors. This consortia is assembled to recognize and leverage the symbiotic relationships among its members, providing an inoculum that is both effective and robust in various environmental contexts. The importance of such a consortia lies in its ability to synergistically enhance the digestion of recalcitrant polymers, ensuring the efficient breakdown of complex polymer mixtures often found in waste products.

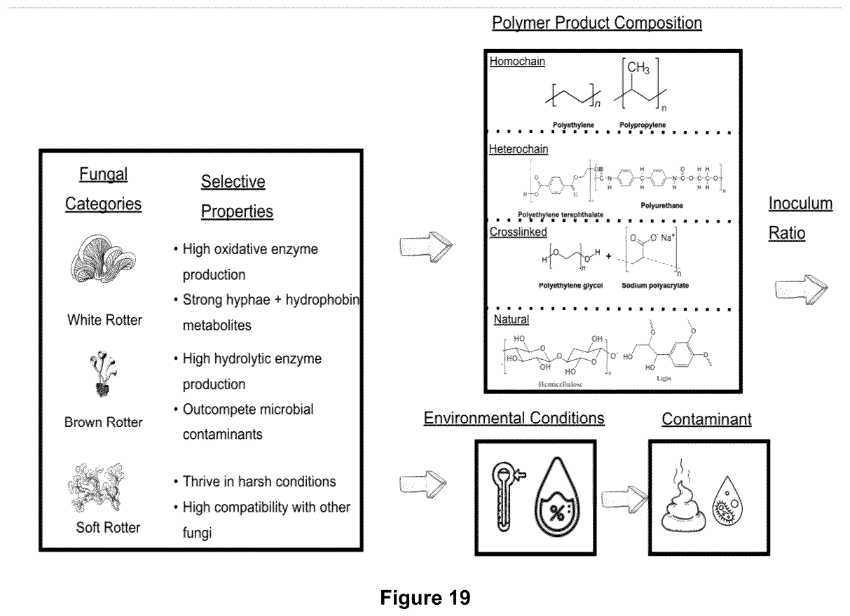

FIG. 19 illustrates the composition of the fungal consortia, comprising two or more preselected fungi. These fungi are chosen based on their ability to create a viable consortia within disposal environments, synergistically enhancing the digestion of stubborn polymeric materials. The consortia is capable of addressing complex polymer products containing multiple types of polymers. The selective properties of consortia members are highlighted, highlighting their strengths in a given product mixture environment. These fungi are selected from a pre-screened catalog, adapted from traits observed in nature, ensuring that each member's enzymatic and metabolic capabilities are optimized for polymer digestion.

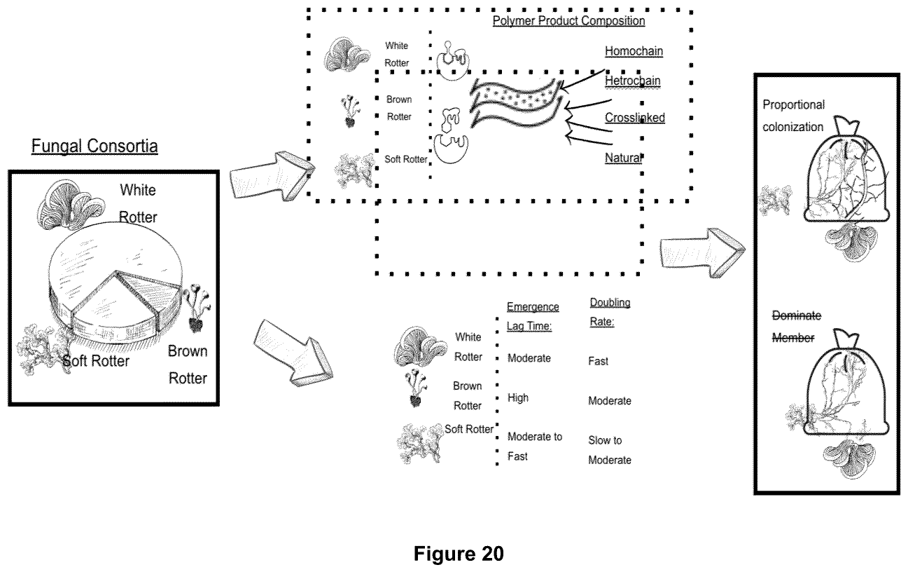

FIG. 20 shows the unique blend of the fungal consortia, demonstrating how it is tuned to target a particular polymer product. For instance, products with a higher proportion of homochains require more fungi that produce oxidative enzymes compared to those with predominantly heterochains and natural polymers. The consortia members are blended in proportions to ensure efficient colonization and digestion. This encapsulated blend is configured to enable emergence and colonization from all members, even at scaled-down inoculum ratios. The ratio is a function of factors such as doubling rate and emergence lag time, emphasizing the complexity of creating an effective species blend with less than a 1:1 inoculation ratio, and in some cases less than a 0.1:1 ratio. The disclosed embodiments ensure a consortia that can effectively digest polymers even at reduced inoculum concentrations.

### Example 8: Species Consortia Inoculum Blend Formulation

Example 8 shows formation of a blend of fungal species that effectively colonize and work synergistically, without outcompeting each other. Each species' lag time from emergence, growth rate, and compatibility were considered and analyzed, and impact the proportions of each species within the inoculum blend to ensure effectiveness. Four Species S and two Species W species were blended in various ratios according to Table 6 below. Each petri was inoculated with a total bead weight of 10 g.

TABLE 6
Species Ratio Blend

| Species | Blend 1 | Blend 2 | Blend 3 | Blend 4 |
|---|---|---|---|---|
| Species W₁ | 25% | 25% | 30% | 20% |
| Species S₁ | 25% | 15% | 10% | 10% |
| Species S₂ | 25% | 25% | 20% | 30% |
| Species W₂ | 25% | 35% | 40% | 40% |

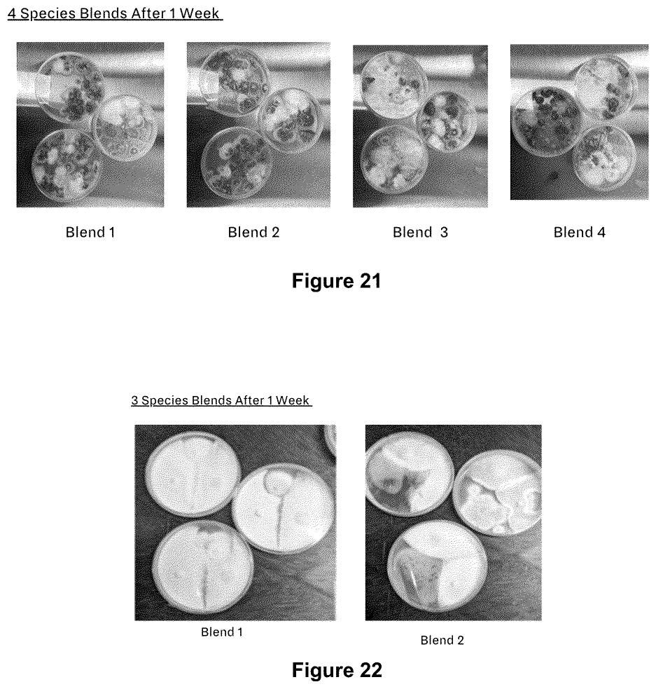

Observations showed that Blends 1 and 2 yielded effective growth from Species S1 and W1, with little or no growth from Species S2 and W2. Blend 3 demonstrated effective growth from all species except Species S2. The results for Blend 4 were inconsistent, however, one replicate showed significant growth from all four species. FIG. 21 includes photographs showing growth of each of the Blends of Table 6 after one week.

The blend was optimized to ensure proportional growth for three Species W and Species S. FIG. 22 includes photographs of fungal growth from these optimized blends, demonstrating balanced and effective growth of all selected species.

### Example 9A: Species Consortia Inoculum Blend Testing

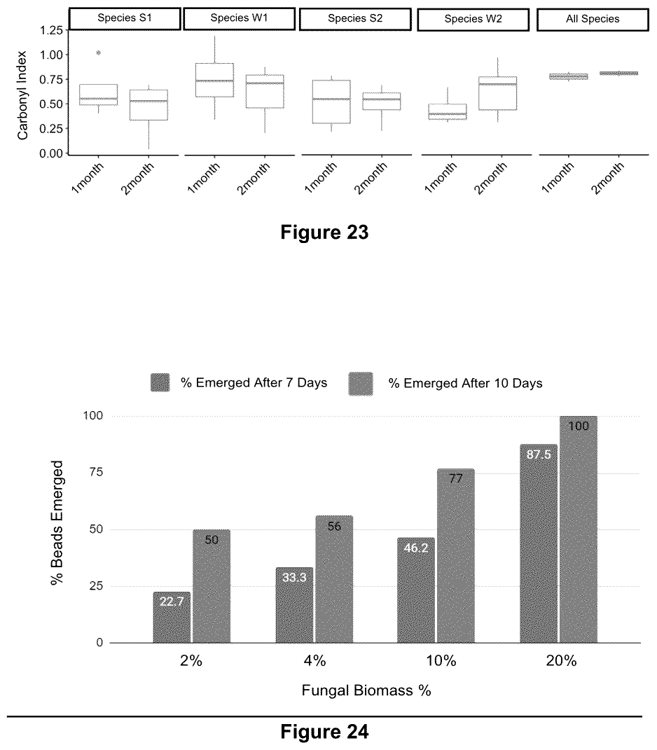

In Example 9, two Species W and two Species S were tested with 10% inoculum on soiled diapers, individually and mixed together into a blend with each other, with 12 replicates for each treatment. The diapers were placed in buckets with their replicates and buried in soil outside. Samples of PE film were pulled after one month and two months for FTIR analysis. FIG. 23 shows the carbonyl index of each species separately and all together. The box and whiskers plot demonstrates that while some individual Species did reach a carbonyl index over 1, the species blend had the most consistent and effective results, showing the synergistic effect achieved by the species consortia. This highlights the importance of species diversity in real-world environments wherein the fungi must compete with biotic and abiotic stressors.

### Example 9B: Species Consortia Inoculum Blend Testing

In Example 9B, the highest performing individual Species from Example 9A were tested individually against the highest performing three-species blend and highest performing four-species blend. Samples were collected after three months for DSC analysis and cut to obtain PP nonwoven, PE film, and PET nonwoven layers. Table 7 shows that Species W3 and W4 demonstrated effective digestion on PE and PP but failed to colonize and digest the PET layer. The three-species blend showed moderate effectiveness for degrading PE, was effective at degrading the PET layer, but did not colonize PP nonwoven. The four-species blend was effective on all three layers, being the most effective on PE and PET, while less effective on PP than W3 alone. Example 9B demonstrates the synergistic results achieved with embodiments of the inoculum with multiple engineer fungal species in a consortium for degrading a suite of polymers while not inhibiting the digestion of other polymers. Species W was more effective on homochains, but less so on heterochains, while Species S was effective on heterochains. The blend of four species was required to digest all three polymers.

TABLE 7
Crystallinity Reductions after 3 Months
Crystallinity Reduction w Species Combinations

| | PE | PP | PET |
|---|---|---|---|
| Species W1 | 79.96% | 31.59% | No Colonization |
| Species W2 | 69.08% | 92.25% | No Colonization |
| Species W1, W2, S1 | 13.14% | No Colonization | 86.20% |
| Species W1, W2, S1, S2 | 96.16% | 35.45% | 88.19% |

In practice, an inoculant comprising a fungal consortia of the disclosed engineered fungi species that have undergone the disclosed treatments to be more accepting of plastic materials as a carbon source for metabolism and be more resilient to abiotic stress has shown to effectively inoculate a soiled diaper at an initial concentration of below 1% fungal biomass to plastic ratio. Preferably, the engineered fungal species and strains and/or consortia of engineered fungal species and strains are engineered to inoculate at an initial mass ratio of no greater than 1:1 fungal biomass to plastic. More preferably, the engineered fungal species and strains and/or consortia of engineered fungal species and strains can inoculate at an initial mass ratio of no greater than 1:10 fungal biomass to plastic. Embodiments have shown to effectively inoculate at an initial mass ratio of between 1:10 and 1:200 fungal biomass to plastic. Unlike known treatment methods that rely on application of enzymes to plastic material, the disclosed embodiments that utilize consortia of engineered fungi species and strains is self-sustaining, wherein the fungal species can effectively continue growing and thereby expressing more of the enzymes effective at using the plastic material as a carbon source for further reproduction and growth.

### Immobilized Fungal Inoculant: Creation, Control, and Integration for Digestion of Polymer-Based Materials

As noted above, in another embodiment, an immobilized composition includes engineered fungal inoculants capable of targeting and degrading polymers found in polymer-based materials. These compositions have been shown to be shelf-stable for storage and critically can be reactivated for use. In one particular embodiment, the composition comprises numerous fungal species, strains or combination thereof, which are maintained in stasis through dehydration or lyophilization. This provides prolonged shelf-life without compromising effectiveness in promoting the digestion of polymer-based materials once reactivated from the immobilized state.

This composition can take the form of a standalone product configured for use as an additive or similar, or be incorporated into various polymer-based materials such as disposable diapers, sanitary napkins, incontinence pads, wound dressings, wipes, and other fluid or solid waste management products. It can be added in the form of encapsulated alginate beads or other substrates, allowing for a precise unit of delivery and protecting the fungi from contamination.

Described herein is a method for forming a shelf-stable fungal inoculum specifically designed for digestion applications. The formulation includes encapsulating filamentous fungi in a stable form with an encapsulation material that enables gradual rehydration and activation in the presence of moisture. This approach ensures that the fungi remain dormant from metabolism until environmental conditions are suitable for their activity, thereby facilitating their use in various scenarios. The fungal species used are primarily filamentous fungi, selected for their robust capabilities and broad applicability to environments with sufficient moisture.

The encapsulation techniques employed herein are important to the stability and effectiveness of the inoculum. The fungi can be encapsulated with or without a polymer matrix and may be immobilized with just a preservative such a trehalose. An anhydrobiosis state is achieved, wherein the fungi are preserved in a life-without-water state. Encapsulated particles prevent premature emergence and are formulated using alginate (1-5%) to create beads. Factors such as biomass percentage, fragment size, and potential nutrients are optimized to ensure the viability and functionality of the fungi. Crosslinking density is adjusted to affect bead strength and porosity, allowing for proper growth and respiration. Ensuring genetic stability at room temperature. Delivery formats can include discrete beads 122, pressed tablets 124, sheets 126 (similar to dryer sheets via fiber deposition), Velcro strips, capsules 128 (similar to laundry detergent capsules or pharmaceuticals), nonwoven sachets, foam deposition articles 130, film deposition articles 132, and absorbent nonwoven assemblies, as shown generally in FIG. 25.

Upon rehydration and activation, the inoculum gradually absorbs moisture through controlled microenvironments created by the composite. The addition of hydrophilic abiotic components within the composition facilitates higher active pulling of liquid moisture, while a mixture of living mycelium fragments and hygroscopic components pulls moisture from the surroundings including water vapor. Spores may be added to the composition for delayed activation and optimal microenvironment. The rehydration process allows the fungal cell walls to safely rehydrate, restarting metabolic processes and enabling hyphal tips to grow and escape the encapsulation matrix. This initiates the fungi's search for food sources in their environment, ultimately leading to effective digestion of waste materials. Table 8 below details the phases of preparation, storage, waste mixture, rehydration, and metabolic activation, highlighting the interplay of biotic and abiotic factors in this innovative inoculum delivery system.

TABLE 8

| | | | Waste | | Metabolic | |
|---|---|---|---|---|---|---|
| Phase | Preparation | Storage | Mixture | Rehydration | Activation | Emergence |
| Biotic | Mycelium grows | The mycelium | Mycelium | Cell wall | Metabolic | hyphal tips grow |
| | and fragments | is put into | remains in | rehydrates; | processes | and escape the |
| | are mixed with | stasis; the | stasis while | fungal fragments | restart, utilizing | encapsulation |
| | the encapsulation | mixture keeps | product is | rehydrate at a | the mixture for | matrix and begin |
| | mixture. | fungi alive but | in use. | safe rate to | nutrients; | sensing it's |
| | | in stasis. | | avoid damage. | | environment in |
| | | | | | | search of a food |
| | | | | | | source. |
| Abiotic | The encapsulation | Shelf-stable | Mixture is | The inoculum | The abiotic | Encapsulating |
| | mixture is | storage with a | either added | mixture absorbs | mixture | material |
| | prepared in a | desiccant or in | to waste | from micro | provides the | becomes weak |
| | wet phase and | a container | product after | environment, | necessary | enough/porous |
| | then dried. | that prevents | use, or | either | nutrients and | enough to allow |
| | | fluid entry. | embedded | hydrophobically | structural | for mycelium |
| | | | into waste | with surrounding | stability during | to escape. |
| | | | product while | liquid, or | rehydration. | |
| | | | moisture is | hydroscopically | | |
| | | | added from | from water vapor. | | |
| | | | user . . . | | | |

### Example 10: Preparation and Testing of Fungal Inoculum

A fungal inoculum of Species W engineered according to the method described above was prepared by harvesting biomass from liquid fermentation and homogenizing it with a 2% sodium alginate mixture. Beads were formed by extruding this mixture into a calcium chloride solution to crosslink. The beads were then coated with trehalose and freeze-dried until moisture was removed. Various concentrations of fungal biomass were tested in the mixture, ranging from 2% to 20%.

FIG. 24 shows the percentage of beads that have emerged after 7 and 10 days from each mixture, showing increasing emergence with increasing biomass. This data illustrates the ability to control emergence through mixture composition. Quality assurance and quality control (QA/QC) were performed on 2% and 4% fungal mixtures as the lower end represents effectiveness at minimal biomass dosing. These mixtures were left to emerge after 14 days, and results showed that after 14 days, over 90% of the beads had successfully emerged. This example demonstrates the effectiveness of the preparation method and the ability to control fungal emergence based on the composition of the inoculum mixture.

The wet formulation includes essential biotic elements, such as fungal mycelium biomass or spores, mixed with a preservant like trehalose to stabilize and protect the fungi. Additional components, including conditioned media, polymer encapsulation matrices, process aids, and secondary nutrients, are incorporated depending on the application, ensuring optimal growth and activity of the fungi in digestion processes. These components are described in greater detail in Table 9 below.

TABLE 9
Wet Inoculum Mixture Components

| Component | Description |
|---|---|
| Primary Biotic | Fungal mycelium biomass or spores in varying concentrations. |
| Element | |
| Secondary Biotic | Conditioned media, which includes biomass, enzymes, polysaccharides, transcription |
| Element | factors, and anti-microbials. These elements support fungal growth and activity, with |
| | transcription factors signaling the fungi to emerge and activate. |
| Encapsulation | Trehalose is used to stabilize and protect the mycelium during dehydration. It forms a |
| Preservant | glassy matrix that encapsulates and maintains the integrity of cellular structures. |
| | Alternatives include alginate, carrageenan, dextran, maltodextrin, sucrose, and |
| | sorbitol, all of which offer various protective properties. |
| Polymer | A natural biocompatible polymer such as sodium alginate, which can be crosslinked |
| Encapsulation | with calcium chloride to form stable beads. Other options include pectin, agar, and |
| Matrix | chitosan, crosslinked with agents like potassium chloride or glutaraldehyde. These |
| | matrices provide structural stability and control the release of the biotic components. |
| Process Aid | A surfactant like Tween 20, which aids in dispersing the components evenly and |
| | maintaining the stability of the formulation. This ensures that the encapsulated |
| | mycelium is evenly distributed within the mixture. |
| Additives | Secondary nutrients or other abiotic components like glucose or sucralose, which |
| | provide additional energy sources for the fungi. These additives enhance fungal |
| | growth and activity once the inoculum is activated. |

Immobilization techniques are employed to dry the wet mixture, preserving the viability and functionality of the fungal components. Methods such as dehydration, freeze drying, lyophilization, and spray drying are used to remove moisture from the mixture, creating a stable, dry product ready for storage and use in various applications. These techniques are described in greater detail in Table 11 below.

TABLE 11
Immobilization Techniques

| Process | Description |
|---|---|
| Dehydration | The process of removing water from the fungal biomass to create a stable, |
| | dry product. This can be done through air drying, oven drying, or other |
| | dehydration methods that preserve the integrity of the biotic components. |
| Freeze Dry | Also known as lyophilization, this technique involves freezing the fungal |
| | biomass and then reducing the surrounding pressure to allow the frozen |
| | water to sublimate directly from solid to gas. This method preserves the |
| | structure and viability of the fungal cells. |
| Spray Dry | A technique where the fungal biomass is dispersed in a liquid medium and |
| | then sprayed into a hot drying chamber. The small droplets quickly lose |
| | moisture, resulting in a fine, dry powder. This method is efficient and |
| | suitable for large-scale production. |

The dry mixture, resulting from the immobilization process, can be used as is or further enhanced by mixing with additional compounds. This includes incorporating other immobilized biotic elements, hydrophilic and hygroscopic compounding elements, filler materials, disintegration materials, and desiccants to form a comprehensive composition tailored for specific digestion and/or degradation applications. The components of the dry inoculum are described in greater detail in Table 12 below.

TABLE 12
Dry Inoculum Components

| Component | Description |
|---|---|
| One or More | Fungal mycelium or spores preserved in a stable, dormant state. These serve |
| Immobilized Biotic | as the active agents once rehydrated. Options include dried mycelium |
| Components | fragments, encapsulated fragment, or spore powders. |
| Hydrophilic | Compounds that attract and retain water to facilitate rehydration. These |
| Compounding Elements | elements ensure a quick activation of the biotic components. Options include |
| | trehalose, alginate, carrageenan, and dextran. |
| Hygroscopic | Compounds that absorb moisture from the environment, aiding in the gradual |
| Compounding Elements | rehydration of the biotic components. These ensure a controlled release and |
| | activation. Options include glycerol, sorbitol, and maltodextrin. |
| Filler Material | Substances used to provide bulk and shape to the tablet or capsule, ensuring |
| | uniform distribution of active components. These materials do not interfere |
| | with the activity of the biotic elements. Options include cellulose, starch, and |
| | polyvinyl alcohol (PVA). |
| Disintegration | Compounds that help the tablet or capsule break down upon contact with |
| Material | moisture, facilitating the release of the active ingredients. These materials |
| | ensure the rapid activation of the fungal components. Options include natural |
| | fibers, microcrystalline cellulose, and croscarmellose sodium. |
| Desiccant | Substances used to absorb excess moisture and maintain dryness within the |
| | packaging, ensuring the stability and longevity of the biotic components. |
| | Options include silica gel, calcium oxide, and molecular sieves. |

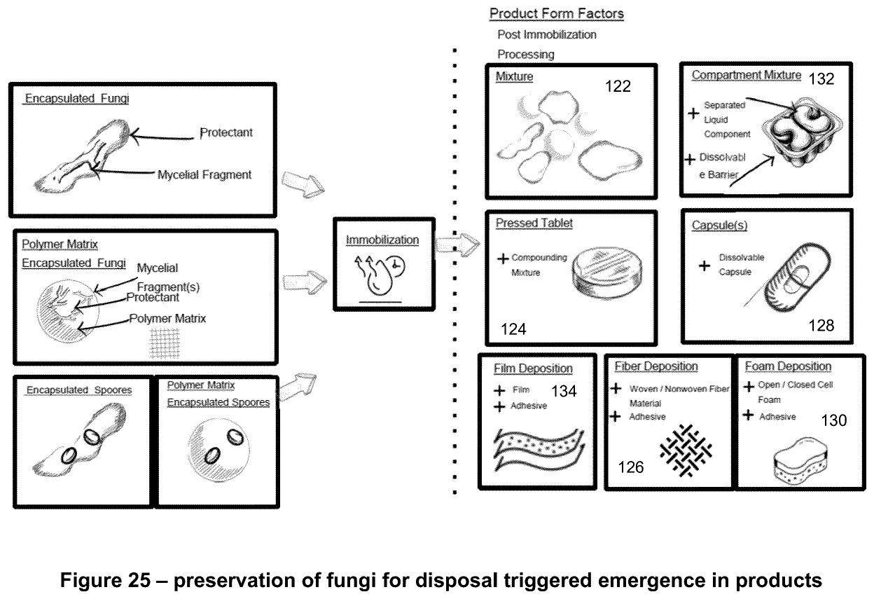

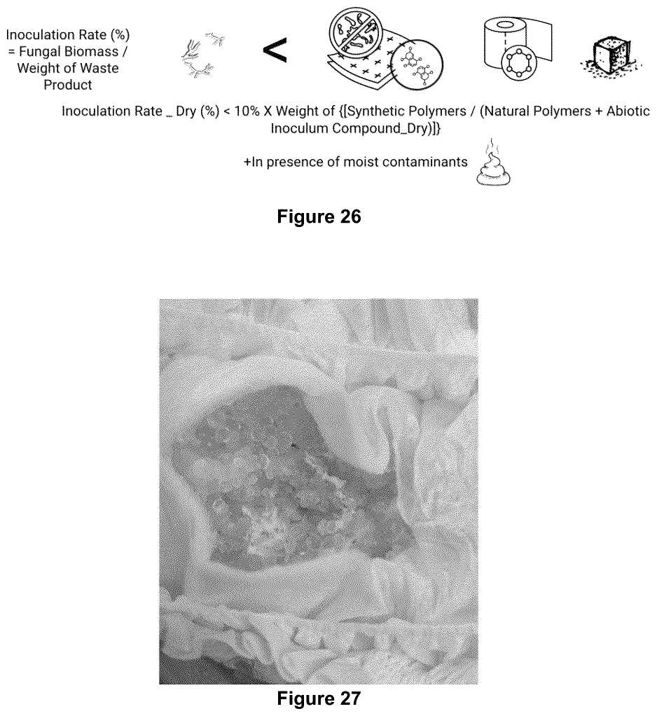

A dried inoculum according to the disclosed embodiments is shelf stable and can be mixed with waste products at lower weight ratios, unlike any known techniques. This approach allows for efficient digestion and/or degradation in real-world scenarios, as opposed to clean lab conditions, where products often consist of a mixture of synthetic and natural polymers contaminated with waste. By considering the complexity of these products, the method ensures effectiveness in practical, non-laboratory environments. For instance, a dry inoculum combined with contaminated polymer products at an inoculation rate of less than 10% of the weight of synthetic polymers, in the presence of moist contaminants, demonstrates significant digestion capabilities, as described in FIG. 26.

### Example 10: Encapsulated Polymer Matrix

In Example 10, creation of a shelf-stable encapsulation of filamentous fungi is established, focusing on the use of a protective polymer matrix and a carefully formulated fungal hyphal mixture. 1% wet fungal biomass from engineered basidiomycete of Species W was employed, although the concentration can reasonably range from 0.1% to 50% within the embodiments. The biomass was mixed with a 2% sodium alginate medium viscosity solution, forming a homogeneous mixture. To create the encapsulation matrix, the alginate-fungal mixture was cross-linked with calcium chloride, resulting in the formation of uniform beads with a wet diameter of approximately 5 mm. These wet beads were then freeze-dried to enhance their shelf stability and stored with a desiccant to prevent moisture absorption during storage. Upon testing, the freeze-dried encapsulated fungi successfully emerged within 14 days when placed on a petri dish and in a soiled diaper environment. The protective polymer matrix and the specific percentage of fungal hyphal mixture were important to maintain the fungi in a stasis state. This composition allowed for slow, controlled rehydration, ensuring that the fungal metabolism reactivated gradually. This process prevented cellular damage from rapid rehydration and protected the fungi from contamination by biotic or abiotic moisture present in the disposal environment.

The above described procedure was repeated with the same components and techniques, and the dried encapsulated beads were placed in a soiled diaper core. FIG. 27 is a photograph showing emergence of fungi from these beads of immobilized encapsulated mycelium in an exemplary waste product environment.

Notably, shelf stable encapsulated fungi were prepared according to this process with non-adapted basidiomycete species for additional confirmation of the effectiveness of the procedure to basidiomycetes.

### Example 11: Encapsulated with Cryopreservant

In Example 11 filamentous fungi were encapsulated without cross-linking, using trehalose as a cryoprotectant to enhance the stability and viability of the fungal biomass. 1% wet fungal biomass from the engineered basidiomycete of Species W was used, however, like in Example 11, a concentration of between 0.1% and 50% can reasonably be employed. Instead of cross-linking, the fungal biomass was mixed with trehalose to create a cryoprotective environment. The mixture was then processed to form a stable composition. Instead of forming beads, the fungal biomass combined with trehalose was integrated with a hydrophilic filler ingredient, ensuring a consistent and protective matrix. This mixture was then freeze-dried to preserve the fungi in a shelf-stable state and stored with a desiccant to maintain its dryness. Upon testing, this embodiment successfully rehydrated when exposed to moisture and combined with the hydrophilic filler ingredient. The presence of trehalose as a cryoprotectant allowed the fungal cells to rehydrate slowly and properly, avoiding damage from rapid moisture uptake. The hydrophilic filler played an important role in ensuring a controlled rehydration process, which is vital for the gradual reactivation of the fungal metabolism.

### Example 12: Encapsulated Mycelium in Dried State for Emergence and Adhesion

In Example 12, the efficacy of trehalose and alginate as encapsulation methods for fungal inoculum was evaluated, specifically focusing on the emergence and adhesion of the encapsulated inoculum on various nonwoven materials. This Example shows that embodiments of an encapsulated/immobilized mycelium in a dried stasis state can be effectively reactivated and thus effectively used, thereby demonstrating its potential for application in polymer matrix encapsulation and trehalose encapsulation forms. The fungal inoculum of 96% alginate and 4% biomass (Species S and Species W) was mixed with trehalose and alginate powders separately to create 20% inoculum concentrations. These mixtures were thoroughly blended to ensure uniform distribution of the inoculum within the powder. The encapsulated inoculum powders were then plated onto agar plates in replicates and labeled accordingly. Different nonwoven materials (loop, back sheet, baby wipe) were selected as substrates for the adhesion experiment. Each material was divided into sections, labeled, and sprayed with adhesive before applying the encapsulated inoculum powders. Control plates without adhesive were also prepared. Upon incubation, the emergence of fungal colonies was monitored and recorded. The results showed that both trehalose- and alginate-encapsulated inoculum powders successfully adhered to the nonwoven materials and demonstrated significant fungal emergence. The efficacy of trehalose and alginate encapsulation methods in promoting fungal emergence was comparable. However, subtle differences were noted in the growth patterns and adhesion efficiency on different substrates. Control plates confirmed that the adhesive spray enhanced the adhesion and subsequent emergence of the encapsulated inoculum.

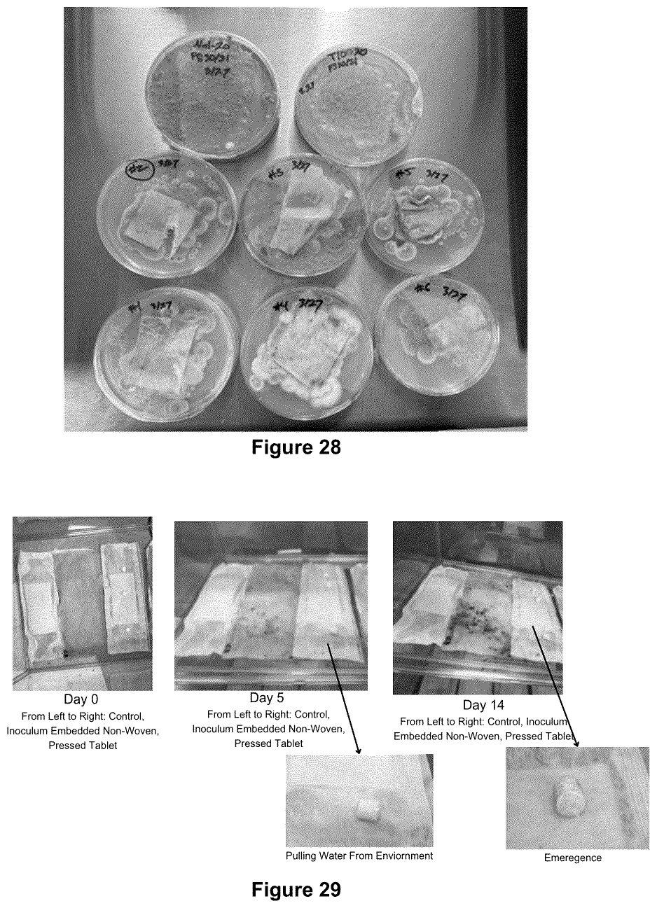

Example 12 thus demonstrates the feasibility of using encapsulated mycelium in a dried and shelf-stable state to promote fungal emergence and adhesion on nonwoven substrates. The results indicate that both trehalose and alginate are effective encapsulation methods, with potential applications in integrating fungal inoculants into polymer-based products for enhanced digestion (see FIG. 28, showing emergence of nonwoven adhered immobilize fungal inoculum).

As noted, the dried inoculum herein described can be formed into various end-product form factors to suit different applications, examples of which are shown in FIG. 25. These include discrete beads or powdered forms 122, pressed tablets 124, dryer sheets, Velcro strips, laundry detergent capsules 134, and nonwoven sachets, as shown generally in FIG. 25. Additionally, the inoculum can be integrated into foam/sponge assemblies 130, absorbent nonwoven assemblies 126, and Listerine strip-like nonwoven ultrasonic bonds 132. These diverse delivery formats enhance the versatility and scalability of the inoculum, allowing it to be effectively used in multiple waste digestion contexts. All of these form factors have shown efficacy for allowing rehydration and activation of the fungal inoculants via gradual rehydration with time release. Additionally, the products comprise nutrients that provide a controlled microenvironment that assists the rehydration process. They may include hygroscopic trehalose for higher active concentration. The presence of hydroscopic components with the living mycelium fragments acts to pull moisture from surroundings. Further, spores may be included for providing delayed activation. The disclosed composition products have shown genetic stability at room temperature when combined with desiccants.

A fungal micro-colony used within the beads can be as small as 0.5 microns, representing the smallest viable mycelial fragment, whereas a mycelial fragment of this size found in nature would not be able to survive without novel engineering of inoculum. By isolating and engineering these micro-colonies to survive and propagate even at such small sizes, the inoculant composition can be effectively distributed over large areas with minimal material, reducing costs and enhancing efficiency. This approach allows for a more controlled and consistent inoculation process, making it highly suitable for industrial and commercial applications in polymer degradation.

In nature, fungal growth typically occurs as interconnected mycelium networks or large, diffuse colonies, originating from spores that spread and colonize vast areas. These natural colonies exhibit uniformity in their development and interaction with the environment. In contrast, the micro-colonies in this inoculant composition are not derived from spores but are engineered and recovered from a biomass harvest of specifically engineered fungi. Each encapsulated micro-colony is isolated, enabling it to develop distinct properties and behaviors. This engineered process results in a plurality of micro-colonies with varied tendencies, allowing them to adapt to their specific micro-environments. This diversity within the micro-colonies creates a more robust and adaptable consortia, enhancing the overall efficacy and functionality of the inoculant composition in digesting polymer materials.

### Example 13A: Emergence of Fungal Inoculum Compositions

In Example 13, a nonwoven material embedded with encapsulated mycelium and pressed tablets containing encapsulated mycelium were prepared, each encapsulated with an engineered Species S. The absorbent pads were hydrated, but no additional fluid was added to the mixture. The combination was then set in an aquarium. FIG. 29 includes photographs showing the rehydration process and emergence of both inoculums after 5 and 14 days, respectively, demonstrating the pulling in of water for rehydration and the subsequent emergence of the fungi.

### Example 13B: Emergence of Fungal Inoculum Compositions

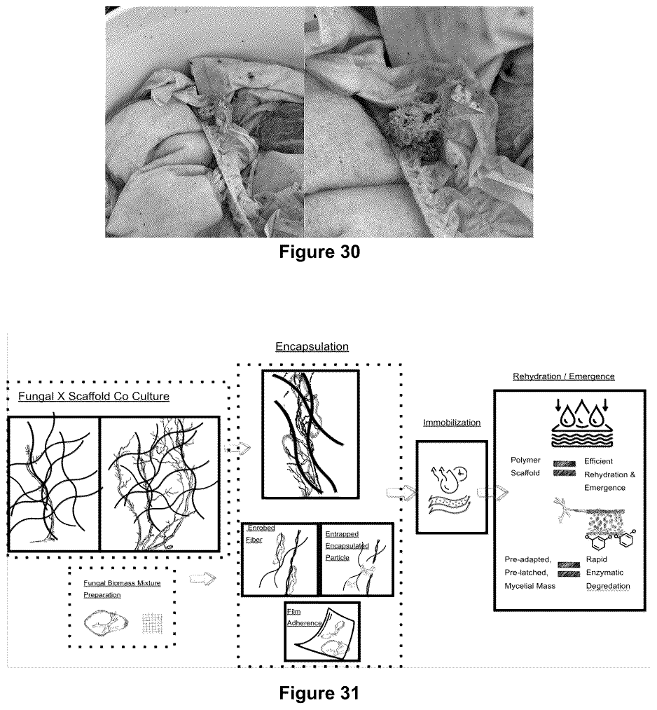

Example 13B tested pressed tablets comparable in composition to those of Example 13A in soiled diapers in an outside bucket environment. After 18 days, the bucket was opened. FIG. 30 includes photographs showing emergence and vigorous colonization of the diaper materials by fungal hyphae.

### Scaffold Encapsulation for Fungal Immobilization

This embodiment concerns a process for growing filamentous fungi on polymer scaffolds, which are then encapsulated to create a shelf-stable product, as shown generally in FIG. 31. The method and resulting article allows the fungi to adapt to various polymer types, enhancing their emergence efficiency and vigor. The encapsulated fungi can be applied to films, nonwovens, foams, and other polymer-based materials.

The scaffold types include nonwoven materials that come into direct contact with fibers or films, enhancing absorbency with encapsulated living organisms. The growth and encapsulation process involves growing filamentous fungi into the nonwoven scaffold, utilizing a novel technique that offers a larger surface area for better adaptation. This process combines polymers to facilitate efficient fungal metabolism.

The scaffold can take various forms, including foams, particles, and actual plastic resins, with fungal spores embedded in the scaffold. Both solid-state and liquid-state encapsulation methods are used to ensure the fungi's viability and effectiveness. This approach allows for diverse applications, including the treatment of films, nonwovens, foams, and other polymer-based materials. The process of growing filamentous fungi into the nonwoven and encapsulating them, whether embedded or not, provides a versatile and efficient method for digestion and/or degradation in various environmental contexts.

### Example 14: Solid State Fermentation with Nonwoven Integration

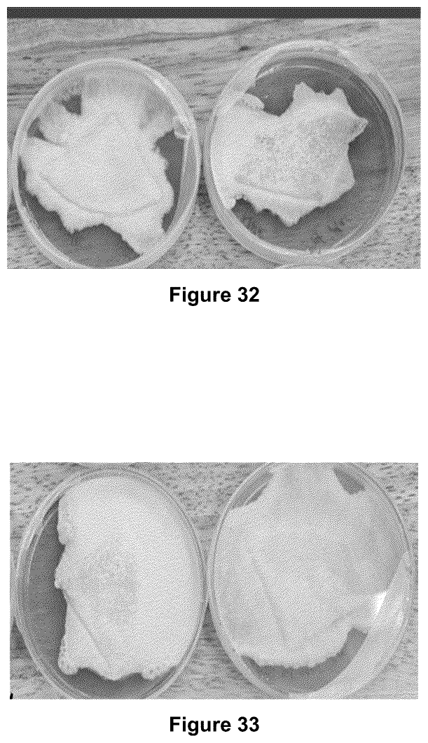

In Example 14, engineered filamentous fungi were colonized using solid state fermentation. The fungi were directed to produce aerial hyphae, which combined with the fibers in a nonwoven material placed on top of the fungal culture. This process ensured that the fungal mycelium integrated thoroughly with the nonwoven fibers, creating a strong physical scaffold. The entire nonwoven sheet, now colonized with fungal mycelium, was then combined with a sodium alginate mixture. This mixture provided additional structural support and protection. The combined sheet was subsequently cross-linked in a calcium chloride solution, forming a robust, immobilized structure. This approach allowed the fungal mycelium to have a physical scaffold for attachment, providing structural support for immobilization and creating a directed microenvironment for controlled rehydration. Upon testing, this variation successfully rehydrated and allowed the fungi to emerge and colonize the material effectively. The enzymatic activity was accelerated as the metabolic processes of the fungi, which were in a state of dormancy during immobilization, were reactivated, promoting rapid digestion of the polymer scaffold. FIG. 32 includes photographs showing emergence of fiber scaffold inoculum of engineered Species W after 7 days.

### Example 15: Enrobing Fibers with Alginate-Mycelium Mixture

In Example 15, instead of solely growing the mycelium through the nonwoven material, a sodium alginate mixture with a specific viscosity and mycelium size was prepared. This preparation ensured that the alginate-mycelium mixture could enrobe the fibers of the nonwoven material thoroughly. The alginate-mycelium mixture was then applied to the nonwoven material, allowing the fungal hyphae to integrate with the fibers. This enrobing process provided a protective layer around the fibers. The entire composition was cross-linked using calcium chloride, resulting in a solid, immobilized structure. This method and resulting article also ensured that the fungi had a physical scaffold for support and a directed microenvironment for controlled rehydration. Upon testing, this variation, like that of Example 14, successfully rehydrated and allowed the fungi to emerge and colonize the material effectively. The metabolic processes of the fungi were preserved during immobilization and reactivated upon rehydration, leading to accelerated digestion of the polymer scaffold via enzymatic activity. FIG. 33 includes photographs showing emergence of enrobed fiber inoculum of engineered Species W after 7 days.

### Example 16: Absorbent Product with Embedded Shelf Stable Fungal Inoculum

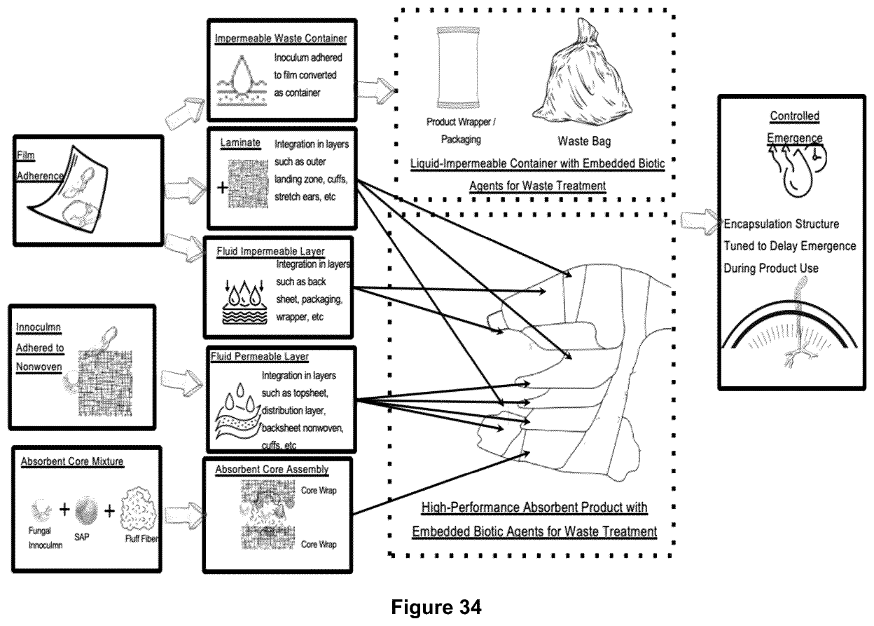

In Example 16, a fluid absorbent product similar to a diaper was constructed with multiple layers, including a liquid-impermeable backsheet, an absorbent core of fluff pulp and superabsorbent polymer wrapped in a core wrap nonwoven, an acquisition and distribution layer, and a polypropylene topsheet, as shown generally in FIG. 34. A control product was prepared with the same construction, but without any immobilized living organisms, incorporating 1 g of superabsorbent polymer and 2 g of films, nonwovens, and adhesives. Several test samples were also prepared for testing the form factors described above: one sample with 1 g of inoculant with engineered fungal Species S in dried alginate bead form added to the core mixture; one sample with 3 g of dried engineered mycelium Species S embedded in the core wrap; one sample with both 1 g of the same alginate beads in the core and 3 g of the same mycelium in the core wrap; one sample with a “landing zone” on the back sheet, which included a polymer film layer, 1 g of encapsulated mycelium, and a nonwoven layer adhered to the back sheet.

To confirm the effectiveness of these absorbent products as usable products, fluid retention and rewet tests were performed on all samples. For fluid retention, the products were submerged in water for one minute, hang-dried for two minutes, and the retained fluid was calculated by subtracting the dry weight from the wet weight. The rewet test involved placing a dry filter paper on the product, applying a 1 kg weight for 10 seconds, and comparing the wet and dry weights of the filter paper to measure moisture retention.

Additionally, the samples were placed in an aquarium box to observe the emergence of fungi, defined as visible growth over 1 cm². The control sample showed no fungal growth, as expected, while samples with encapsulated engineered fungi exhibited varied emergence times for different form factors: 11 days for alginate beads, 5 days for embedded mycelium, and no growth for the outer attached layer. Results are summarized in Table 13 below.

TABLE 13
Absorbance and Emergence Time of Embed Fungal Inoculum

| | | | Time to |
|---|---|---|---|
| | Retained | Rewet | Emerge |
| Sample | Liquid (g) | (g) | (days) |
| Control | 43.79 | 0.54 | N/A |
| 1 g Alginate Beads in Core | 52.26 | 0.56 | 7-10 |
| 3 g Immobilized Fungi Embedded in | 43.48 | 0.53 | 5 |
| Core Wrap | | | |
| 1 g alginate beads in core, 3 g | 53.52 | 0.56 | 5-7 |
| immobilized fungi in core wrap | | | |
| 1 g Encapsulated Fungi on Landing | 44.5 | 0.51 | 4-5 |
| Zone | | | |

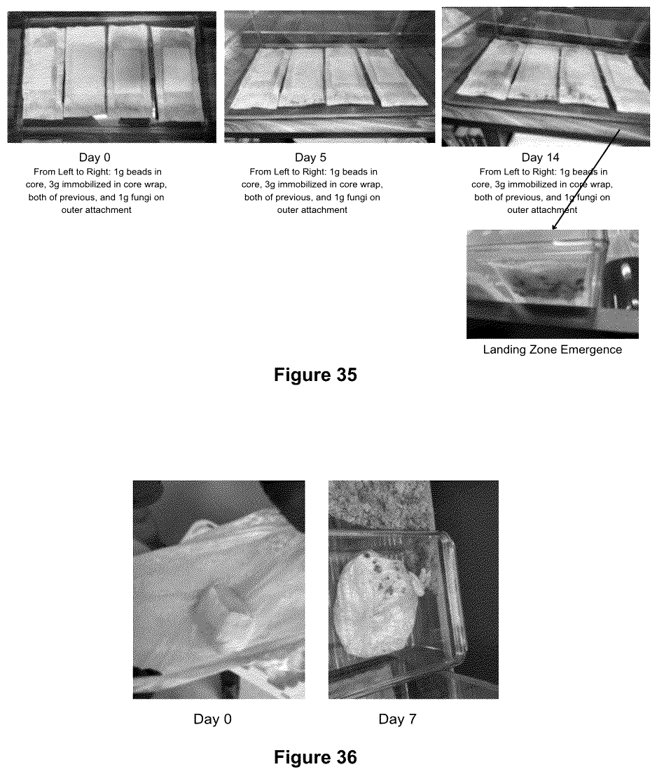

The fluid retention results showed that the control sample absorbed 43.79 g of liquid. Samples with fungi embedded into a polymer layer absorbed similar amounts of fluid, within a 2% margin. Samples with added encapsulated mycelium beads absorbed approximately 6 g more, indicating that encapsulated fungi in nonwovens inside and outside the absorbent product did not affect absorbency, but the addition of alginate beads enhanced fluid retention by a ratio of 6:1. Rewet tests showed that all samples retained moisture similarly, ranging from 0.5 to 0.56 g, indicating that the fungal inoculum did not impact the product's ability to maintain dryness. In terms of emergence, the control sample did not exhibit any fungal growth, while the sample with embedded mycelium showed emergence in about 5 days, compared to 11 days for the alginate beads, and no growth was observed in the chassis of the outer attached layer. These results demonstrate the feasibility of embedding immobilized living organisms in absorbent products without compromising their absorbent properties, while also enabling effective fungal growth. FIG. 35 includes photographs showing emergence of fungal inoculum from engineered Species S from the absorbent product.

### Example 17: Liquid-Impermeable Container with Embedded Biotic Agents for Waste Treatment

In Example 17, 1 g of non-encapsulated immobilized engineered Species S mycelium was adhered to a plastic film bag. A size 4 diaper, with no added mycelium, was wetted with 200 ml of a saltwater and urea mixture. The wet diaper was then placed inside the mycelium-embedded bag. After 7 days, emergence was observed with fungal growth appearing on both the bag and the outer layer of the diaper. FIG. 36 includes photographs showing emergence from film container from Species S inoculum. The results of Example 17 indicate that fungi can be embedded in waste bags or other packaging commonly used in feminine care products, for example, and yield effective colonization and digestion. By incorporating a fungal inoculum into the waste stream post-use, the fungi utilize the soiled product for rehydration and emergence, facilitating digestion within the disposal environment.

In one embodiment, a method to control the rehydration and emergence of immobilized fungal inoculants in absorbent products includes using specific conditions for rehydration of the immobilized fungal inoculant. The method allows for the use of fungal inoculants, where their rehydration can be strategically controlled. The immobilized fungal inoculant is typically held in stasis and then configured in such a manner that it is rehydrated with the soiling of an absorbent product. This could be in an environment where the hydrogel is swelling within the absorbent core of a diaper, or embedded into a nonwoven substrate that draws liquid to the encapsulated material.

Hydrogels are utilized as the medium for fungal inoculant immobilization due to their unique physical properties. As three-dimensional cross-linked networks of polymer chains, hydrogels have the capacity to absorb and retain substantial quantities of water within the interstitial spaces between the polymer chains. These properties, along with their softness, hydrophilicity, super-absorbency, viscoelasticity, biodegradability, and biocompatibility, make hydrogels ideal for biological applications, including in the disclosed embodiments.

Notably, the application of smart or stimuli-responsive hydrogels represents a significant variation. Smart hydrogels are sensitive to specific environmental changes and show responses by changing their shape or volume when exposed to these conditions. This sensitivity to stimuli can be categorized as internal or external, depending on their source at the time of application to the hydrogels in vivo. The external stimuli that can be used to control the rehydration and emergence of the immobilized fungal inoculants include physical conditions such as temperature, pressure, light, electric field, magnetic field, and ultrasound irradiation. Alternatively, the hydrogels can be configured to be responsive to chemical stimuli such as pH, ionic strength, and CO₂.

pH-sensitive hydrogels can be designed to only allow rehydration and emergence of the immobilized fungal inoculants at specific pH values. For instance, the hydrogels can be configured to not react in the presence of water but to only rehydrate and activate the fungal inoculants when exposed to urine. This approach makes use of immobilized fungal inoculants in consumer absorbent products, such as diapers, sanitary pads, and incontinence pads, both safe and practical.

The hydrogel coatings used herein serve to ensure the immobilized fungal inoculants are only rehydrated under specific conditions, enabling the safe and effective use of these fungi in consumer absorbent products. By offering a precise control over the rehydration and re-emergence of fungal inoculants, the product brings a higher level of sophistication and specificity to the application of fungi in the field of absorbent products.

Also provided is a method of embedding immobilized fungal inoculants within nonwoven or foam substrates. These substrates have fluid handling capabilities due to their capillarity and permeability, properties that ensure liquid travels to the immobilized fungal inoculant effectively. In this context, capillarity refers to the phenomenon where liquid ascends in the substrate against gravity due to intermolecular forces. Similarly, permeability defines the substrate's ability to allow liquids to pass through it. Nonwoven or foam substrates with high capillarity and permeability absorb fluid efficiently, ensuring that it reaches the encapsulated fungal inoculants, triggering their rehydration and emergence. This strategic positioning within the substrate and subsequent hydration only when exposed to specific fluid conditions, like urine, provides a highly targeted and efficient mechanism for the fungal inoculants' activation, further enhancing the safety and utility of this invention in consumer absorbent products.

Water absorbent capacity (WAC) is governed by the relationship:

((osmotic pressure\hat{ }2)+affinity)/rubber elasticity, wherein.
Osmotic Pressure: In the present formulation, the osmotic pressure is influenced by the ion concentration inside the hydrogel and the surrounding fluid. Sodium alginate is a polymer that forms a gel in the presence of calcium ions (from CaCl₂)). The sodium ions from the sodium alginate and the calcium ions from the CaCl₂) contribute to the ion concentration inside the hydrogel. The difference in ion concentration between the inside of the hydrogel and the surrounding fluid creates an osmotic pressure that drives water absorption.
Affinity: The affinity in the present formulation is determined by the hydrophilic groups present in the hydrogel. Sodium alginate is a hydrophilic polymer, meaning it has a high affinity for water. This high affinity contributes to the water absorption capacity of the hydrogel. The fungal hyphae may also contribute to the hydrophilic nature of the hydrogel, depending on the specific species of fungi used.
Rubber Elasticity: The rubber elasticity the present formulation is a function of the crosslinking density. In your case, the crosslinking is provided by the calcium ions (from CaCl₂)), which crosslink the sodium alginate to form a gel. The density of these crosslinks determines the rubber elasticity of the hydrogel. By adjusting the concentration of CaCl₂), you can control the crosslinking density and, therefore, the rubber elasticity of the hydrogel.

The osmotic pressure, created by the ion differential, is the primary driver of swelling, and it increases with the square of the ion concentration difference. Affinity, related to hydrophilic groups, plays a lesser role. Rubber elasticity, determined by the degree of crosslinking, resists swelling. The granule will swell until the osmotic pressure, affinity, and rubber elasticity are balanced.

Synthetic Absorbent Hydrogel: Synthetic polymer created through polymerization of monomers.
Synthetic super absorbents undergoes polymerization from monomers, such as NaPA, in multiple polymerization steps, and is crosslinked with polymer.

Natural Absorbent Hydrogel: Natural polymer that undergoes ionic or chemical crosslinking.
Polymer of natural original such as alginate (or Cellulose, Chitosan, Agar, Pectin, Starch, Hyaluronic Acid, Carrageenan, Guar Gum, Xanthan Gum), grafted or crosslinked (ionic or chemical crosslinkers) to enhanced water absorbing capabilities.

When Sodium Polyacrylate (NaPA) crosslinked with 5% Polyethylene Glycol (PEG) is mixed with sodium alginate crosslinked with CaCl₂), an ion exchange occurs. Na⁺ ions from the NaPA replace Ca²⁺ ions in the alginate network. This exchange reduces the crosslinking density in the alginate (as Ca²⁺ ions create stronger crosslinks than Na⁺ ions), thereby decreasing its rubber elasticity and allowing it to swell more. Meanwhile, the SAP becomes more crosslinked due to the influx of Ca²⁺ ions, increasing its rubber elasticity and reducing its swelling capacity.

The change in osmotic pressure (which increases with the square of the ion concentration difference) significantly impacts the system's overall absorbency. While the SAP becomes more elastic and less absorbent, the alginate, by losing its crosslinking, swells more efficiently. This ion exchange optimally utilizes the strengths of both the natural hydrogel (alginate) and the synthetic hydrogel (SAP). The alginate can absorb more fluid, making the system more efficient, while the SAP, with increased elasticity, provides structural support. This balanced approach reduces the amount of SAP needed, leveraging the natural polymer hydrogel's enhanced swelling capacity.

### Example 18: Fluid Retention with Varying Amounts of Dried SAP and Alginate Encapsulated Hydrogels

In Example 18, conditions involving varying amounts of dried superabsorbent polymer (SAP) and dried alginate encapsulated hydrogels and combinations thereof were tested for fluid retention. Three different samples were prepared: one Sample A with 1 g of alginate hydrogels only; one Sample B with 1 g of SAP only; and Sample C sample with a combination of 1 g SAP and 1 g alginate beads. Each mixture was placed in a tray, 100 g of 1% NaCl solution was added, and tray was placed in zip-lock bag. The samples were allowed to contact the fluid for three different time intervals: 2 minutes, 1 hour, and 4 hours. Each mixture was tested in triplicate. After the designated contact time, the mixtures were strained for 2 minutes, and the fluid retention was calculated as the strained wet weight minus the original dry weight.
Sample A: retained 5.8 grams of fluid after 2 minutes, 4.6 grams after 1 hour, and 4.4 grams after 4 hours.
Sample B: consistently absorbed and retained 40-45 grams of fluid, regardless of the contact time with the water.
Sample C: retained 49.1 grams of fluid after 2 minutes, 78.7 grams after 1 hour, and 98.7 grams (nearly all) of the fluid after 4 hours.

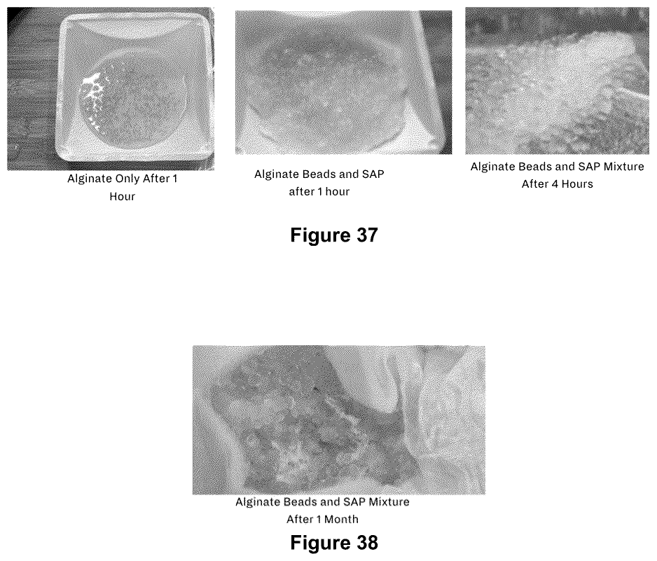

The results show that the mixture of SAP and alginate beads (Sample C) demonstrated an unexpected synergistic effect, significantly enhancing fluid retention over time. While the SAP alone retained a consistent amount of fluid, the addition of alginate beads dramatically increased the total fluid retention, especially over extended periods. This indicates that combining a synthetic hydrogel like SAP with a natural hydrogel like the alginate encapsulated hydrogels can substantially improve the absorbency and fluid retention properties of absorbent products, making them more effective for prolonged use. FIG. 37 includes photographs showing results of Example 18.

### Example 19: Field Testing of Alginate Encapsulated Hydrogels in a Diaper

In Example 19, to test the effectiveness of alginate encapsulated hydrogels in a real-world setting, a size 4 diaper was used. The diaper, soiled with 225 g of urine, was cut open to access the absorbent core. One gram of dried sodium alginate beads, crosslinked with calcium chloride and measuring 0.9-1.1 mm in diameter when dry, was added to the core.

Over the course of one month, the beads swelled to greater than 3 mm in diameter. In comparison, when the same beads were mixed with just water or urine without access to the superabsorbent polymer (SAP), they only swelled to 1.5-1.6 mm. This significant difference in swelling indicates that the presence of SAP in the soiled diaper core facilitated the rehydration and swelling of the alginate beads.

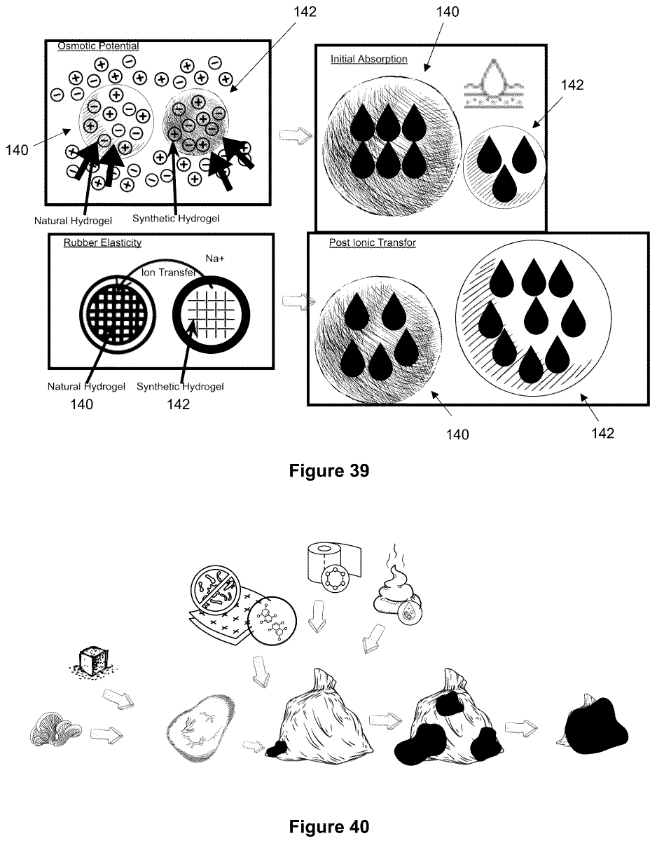

The moisture rehydrating the beads was driven by the interaction with the swollen SAP, allowing the natural polymer hydrogels to access moisture within the SAP. This interaction enabled the beads to swell beyond the typical osmotic pressure equilibrium due to ion exchange from the SAP. This field test demonstrates that embedding alginate beads in the absorbent core of a diaper post-soiling effectively utilizes the moisture retained in the SAP to maximize the swelling and functionality of the hydrogels. FIG. 38 is a photograph showing absorbency of the natural and synthetic hydrogel mixture of Example 16 after 1 Month in the diaper. FIG. 39 depicts this unexpected synergistic effect of combining a natural hydrogel 140 and synthetic hydrogel 142.

### Example 20

In Example 20, an encapsulated engineered fungi/alginate mixture was prepared. This mixture was designed to encapsulate the fungi in a protective alginate matrix, thus immobilizing them while preserving their viability and potential for re-emergence under specific conditions.

The encapsulated fungi/alginate mixture was then embedded onto a cellulose-based nonwoven substrate. This substrate was selected for its fluid-handling capabilities, particularly its permeability and capillarity, ensuring effective fluid absorption and transportation to the encapsulated fungi.

After embedding, the mixture-substrate composition was dehydrated, causing the fungi to enter a state of stasis, further reinforcing their immobilization. This dehydration process effectively preserved the embedded fungi/alginate mixture, readying it for future rehydration and emergence. Subsequently, this dehydrated mixture-substrate composition was subjected to rehydration under controlled conditions. These conditions were specifically designed to simulate the type of fluid exposure the composition would encounter in an absorbent product application. Following successful rehydration, it was observed that the previously immobilized fungi emerged from their encapsulated state and began to colonize a provided agar plate. This colonization demonstrated not only the successful re-emergence of the fungi from their encapsulated and immobilized state but also their continued viability and functionality post-rehydration.

This example shows the controlled rehydration and emergence of immobilized fungi under specific conditions. This capacity can be harnessed to develop consumer absorbent products with advanced functionality, leveraging the potential of fungi in materials digestion or degradation.

In yet another embodiment, a process of encapsulating and immobilizing preselected fungi for commercial use in digesting recalcitrant long-chain carbon materials, particularly plastics and other polymer-based waste, is provided. This process is a natural progression after the scaling up of preselected fungal species in the bioreactor.

Once the mycelial growth has reached its maximum expansion, the fungal hyphae are subjected to a bioprocessing step, typically involving homogenization, and possibly filtration, to create a uniform biomass. This homogenized biomass is then mixed with a polymer solution, such as alginate, to form a stable, manageable material for further processing.

The fungal hyphae-alginate mixture can thereafter be mixed with a curing solution to form a hydrogel, which is then subjected to spray drying. The spray drying can be directed either into a designated collection area for future use, or directly onto a substrate where the fungi will act upon. Alternatively, the fungal hyphae-alginate mixture can be extruded into a solution of calcium carbonate to form beads, which are subsequently recovered.

The beads or the spray-dried material, now containing immobilized fungi, can be further treated for enhanced shelf-life and resilience. This treatment may include coating with trehalose or other similar compounds known for their protective properties during desiccation. This further treatment step is optional, and could also be omitted depending on the intended final use and storage conditions of the product.

The treated or untreated fungal product is then dehydrated, either by freeze-drying or through heat in an oven, with temperatures maintained below species temperature threshold to prevent denaturation. This dehydration process significantly reduces the weight of the product, by 10-75 times, enhancing its ease of handling and reducing transport costs.

The dehydrated fungal product is packed for storage and distribution. The encapsulated and immobilized fungi are shelf-stable and retain their ability to digest recalcitrant long-chain carbon materials, ready for commercial deployment in waste management applications.

A process for adapting and optimizing developed methods for small scale waste digestion into those for large scale industrial applications begins with the collection and aggregation of waste products that consist of a mixture of polymer-based materials and organic waste, such as food waste, yard waste, and other organic matter. The waste streams can be gathered from various sources, including municipal waste management facilities, manufacturing plants, and other industries producing polymer-based waste.

The collected waste materials are then subjected to a similar process as described in the previous inventions, with the introduction of preselected fungal inoculants. The selected fungal species, adept at digesting specific polymer-based materials, are introduced into the waste mix. The industrial-scale application considers a larger volume of waste materials and thus requires larger quantities of fungal inoculants.

The waste materials are placed in a designated waste receptacle, which in this case would be an industrial-scale bioreactor or a similar large-scale waste processing system. An optimal environment for fungal growth and digestion is created and maintained within this system by controlling factors such as temperature, humidity, pH, and aeration.

Additionally, process aids such as nutrient sources, biological enzymes, chemical acids, or other additives can be introduced to enhance the digestion properties of the rehydrated fungal inoculant.

The resulting biomass can then be harvested for further use, such as composting, soil amendment, or as a feedstock for renewable energy production like biofuel or biogas generation.

Ultimately, this process provides an efficient, sustainable, and scalable solution to industrial waste management by leveraging the capabilities of preselected fungi to digest recalcitrant long-chain carbon materials, thereby reducing the environmental footprint of polymer-based waste.

### 1. Pre-Use Incorporation

In this embodiment, a fungal inoculant is integrated into the polymer product itself during its manufacturing phase in one or more of the following ways:
A fungal inoculant is incorporated into the core mixture of absorbent product: The method involves integrating the fungal inoculant directly into an absorbent core material.
A fungal inoculant is included as a component in the mixture of the product's material components.
A fungal inoculant is incorporated within a substrate from offline processing via mixing with the substrate material during a separate processing phase.
A fungal inoculant is included in its non-dehydrated form.
A fungal inoculant is incorporated into an external to product which requires activation. Here, the inoculant is placed externally to the product in a frangible bead, powder or similar structure or form which can be activated by the consumer when needed, such as by contacting with fluid.

### 2. Post-Use Incorporation

In this alternate embodiment, the consumer or waste receptacle introduces a fungal inoculant into contact with a used polymer product at the time of disposal. This can occur via numerous methodologies or actions, all of which are straightforward for everyday use.
Consumers may directly apply the inoculant to a used polymeric product via one or more different form factors including, but not limited to, frangible beads, capsules, wipes and powder. This proactive engagement with the degradation process can facilitate a more personalized and controlled approach to waste management.
Alternatively, the consumer may introduce the fungal inoculant into a waste receptacle or bag designed to hold one or more of the used product(s) after use. This process involves depositing the inoculant into the receptacle where it interacts with the used polymeric product to commence the degradation process.
In another embodiment, the waste receptacle or bag itself has capabilities to disseminate or contain the inoculant. These containers can be pre-loaded with the fungal inoculant, ready to initiate the degradation process once the used product is introduced. This integrated approach minimizes the need for user interaction and maximizes convenience.
In yet another embodiment, the fungal inoculant may be incorporated at any stage in the waste management stream. In these scenarios, waste management facilities or systems could provide the necessary inoculant to the used products, further reducing the responsibility on the consumer.

As discussed generally above, also provided is a superabsorbent hydrogel formulated to incorporate a fungal inoculant, with the unique characteristic of mimicking the properties of superabsorbent polymers (SAPs). This hydrogel product exhibits high swelling capabilities, rapid hydration rates, and excellent fluid retention capacity, all while harboring a fungal inoculant for bioactive applications. The inventive embodiments described herein inherently encompass a novel biodegradable superabsorbent hydrogel formulation as well.

The hydrogel's superabsorbent nature is derived from a specialized formulation involving optimized cross-linking density, careful polymer selection, incorporation of ionizable groups, and advanced processing techniques. Incorporation of a fungal inoculant is central to the efficacy. The fungal strains are chosen based on the desired bioactivity. They are embedded within the hydrogel matrix, wherein they remain dormant until rehydrated. The superabsorbent nature of the hydrogel ensures a controlled and sustained rehydration of the fungal inoculant, facilitating its bioactivity under specific conditions.

Further, the size and dispersion of discrete fungal hydrogels provide optimal available surface area to enhance the: absorption speed, and total absorption capacity of embedded hydrogels. This leads to both an effective method for rehydration of fungal inoculant, and provides an alternative source of absorption capacity crucial for the performance of absorbent products such as diapers. In one embodiment, the SAP can be fully replaced by this novel fungal hydrogel to achieve performance characteristics of an absorbent product.

This inventive product combines the superabsorbent characteristics of SAPs and the bioactivity of fungal inoculants to create a versatile and environmentally friendly material. This superabsorbent hydrogel with fungal inoculant can be tailored for a wide range of applications, including, but not limited to, agriculture, bioremediation, wastewater treatment, and personal care products.

Other embodiments concern a method for integrating a fungal inoculant into the manufacturing process of absorbent products. This innovative process is designed to be seamlessly incorporated into existing manufacturing lines, specifically those utilizing a core forming assembly.

The process begins with the preparation of immobilized fungal inoculant. The fungal inoculant is prepared in a form that is compatible with the existing machinery, specifically, as particles with a size range similar to that of superabsorbent polymers (SAPs), typically between 0.5 mm-1.5 mm with an average size of less than 1 mm. This size compatibility allows the fungal inoculant to be handled and processed in a similar manner to SAPs, facilitating its integration into the manufacturing process.

The immobilized fungal inoculant is then loaded into a hopper, which is positioned upstream of the core forming assembly. This hopper is specifically dedicated to the fungal inoculant, ensuring its accurate and controlled addition to the absorbent product.

The fungal inoculant is then mixed with the pulp fiber mixture. The ratio of fungal inoculant to pulp fiber is carefully calibrated to ensure sufficient fungal activity for the digestion of the polymer product, while maintaining the absorbent properties of the final product.

After thorough mixing with the pulp fiber, the combined mixture is transferred to the core forming assembly. Here, the mixture is placed into a rotating drum, where it is formed into the absorbent core of the product.

This process is designed to ensure that the temperatures experienced by the majority (>90%) of the fungal inoculant particles do not reach levels that would kill the fungi in stasis. This is important for maintaining the bioactivity of the fungal inoculant in the final product.

The embodiments provide unique integration of a bioactive fungal inoculant into the manufacturing process of absorbent products. The fungal inoculant is specifically designed to be compatible with existing core forming machinery, either alone or in combination with SAPs. This compatibility allows for the production of absorbent products with enhanced biodegradability, without requiring significant changes to existing manufacturing processes.

This inventive process provides a practical and efficient method for incorporating fungal inoculants into absorbent products, contributing to the development of more sustainable and environmentally friendly absorbent products.

In an alternative embodiment, the fungal inoculant is embedded and immobilized in a carrier nonwoven substrate. This substrate can be applied between any of the layers of an absorbent product, providing a versatile method for integrating the fungal inoculant into the product.

The process begins with the preparation of the inoculant patch. The fungal inoculant is embedded in a nonwoven substrate, forming a roll of material that can be cut into patches of the desired size. This roll is prepared in advance and can be stored until needed, providing a ready supply of inoculant patches for the manufacturing process.

When it is time to integrate the inoculant into the absorbent product, the roll is unspooled and the patches are cut to the appropriate size. This cutting process can be automated to ensure consistent patch size and to streamline the manufacturing process.

Once the patches are cut, they are picked up by a vacuum rotating drum. This drum grabs the patch and rotates it towards the partially assembled absorbent product. The use of a vacuum rotating drum ensures precise placement of the patches and minimizes the risk of misalignment or displacement during the application process.

The patch is then applied to the absorbent product. This application can be facilitated by the use of a tackifier or adhesive, which helps to secure the patch in place. The choice of tackifier or adhesive can be tailored to the specific requirements of the absorbent product, taking into account factors such as the materials used in the product, the desired product performance, and the environmental conditions in which the product will be used.

Following the application of the patch, a further layer (either a film or nonwoven) is applied over the patch in the absorbent product. This layer serves to hold the patch in place and to protect the fungal inoculant from damage or displacement during the remainder of the manufacturing process and the subsequent use of the product.

This alternative embodiment of the invention provides a practical and efficient method for incorporating fungal inoculants into absorbent products. By embedding the inoculant in a nonwoven substrate and applying it as a patch, the invention allows for the precise placement of the inoculant within the product, contributing to the development of more sustainable and environmentally friendly absorbent products.

### Shelf-Stable Delivery of Living Organism, Discrete Capsule, and Associated Form Factors

In another embodiment, a particle delivery system for shelf-stable living organisms is configured for integration into existing absorbent product manufacturing processes. The system leverages hydrogel particles, enabling seamless mixing with various species post-drying/curing for targeted application in absorbent products. These embodiments provide flexibility for capsule-like final packaging, resembling dry detergent pods, and gel suspension options for post-use applications. This approach allows for the customization of digestion agents, tailored to specific consumer needs and environmental conditions.

This embodiment utilizes a controlled release system using hydrogels for the delivery of shelf-stable living organisms. These hydrogels in a dehydrated state are preferably incorporated within absorbent products made from polymers, such as diapers or sanitary napkins, where they are positioned to encounter fluid post-use. Upon contact with liquid, the hydrogels rehydrate, triggering the emergence of the encapsulated living fungal organisms. This controlled release mechanism ensures that the activation of the digestion agents occurs only in the presence of moisture, thereby aligning the system's activation with the product's functional lifecycle.

The encapsulation of these living organisms is achieved through various techniques, with alginate microparticles being particularly preferred. These microparticles provide a stable environment for the fungi, protecting them until rehydration. Additionally, the system allows for the incorporation of extra nutrients and enzymes into the formulation, enhancing the effectiveness of the fungal inoculants upon activation. This process aids robust digestion process once the hydrogels are rehydrated. Further, the formulation of these particles can be adjusted to include delayed-release coatings in some embodiments. The coatings are engineered to prevent premature activation of the fungi, ensuring that they only become active well after the product's use, thereby maximizing their digestion potential.

A key feature of these embodiments are compatibility with a wide variety of existing absorbent product designs. The discrete capsules, formulated as alginate microparticles or similarly encapsulated hydrogels, can be integrated seamlessly into the structure of these products. Positioned strategically, these capsules remain inert and unobtrusive prior to and during the product's use. However, once exposed to moisture, they activate, contributing to the digestion of the product. This integration is a significant advancement, as it enables the addition of digestion functionality to a wide range of products without altering their core design or user experience.

In another embodiment, shelf-stable living organisms are embedded into a web substrate, creating an advanced system for efficient encapsulation and rehydration. In one particular embodiment, fibrous materials are impregnated with alginate, ensuring adherence and curing for effective encapsulation. In an alternative embodiment, encapsulated beads are imbedded into textiles, with options for microcapsule functionalization or adherence. This method delivers living organisms for digestion, utilizing fibrous scaffolds for enhanced efficiency and effectiveness in absorbent products and other applications.

Additionally, shelf-stable living fungal organisms can be embedded into a nonwoven web substrate which serves as an efficient scaffold for encapsulation and rehydration. This embodiment utilizes advanced techniques such as screen printing, slot coaters, and microgravure roll-to-roll printing. These methods precisely impregnate the fibrous nonwoven materials with alginate, a biocompatible and biodegradable hydrogel, ensuring an effective encapsulation of the fungal organisms within the fibrous matrix. This process not only provides a stable environment for the fungi but also ensures their immediate contact with the nonwoven scaffold upon rehydration, which is critical for accelerating their growth and digestion activity.

In another embodiment, the fibrous nonwoven material may be impregnated with alginate. This process is akin to screen printing, wherein the alginate, mixed with the fungal inoculant, is uniformly distributed across the nonwoven fabric. This distribution allows for a controlled and efficient encapsulation of the fungi, ensuring that they are evenly dispersed throughout the material. After impregnation, the alginate undergoes a curing process, solidifying the encapsulation and rendering the fungi in a dormant yet viable state. This curing can be achieved through various known techniques, including UV curing or heat treatment, providing flexibility in the manufacturing process.

In yet another embodiment, slot coating or microgravure printing processes are utilized. These techniques offer precision in the application of the alginate-fungi mixture, ensuring consistent and effective encapsulation across various types of nonwoven materials. Upon rehydration, the fungi are in immediate contact with the nonwoven scaffold. This proximity provides an ideal environment for the fungi to rapidly rehydrate and commence their growth and digestion processes. The scaffold not only supports the fungi physically but also aids in the efficient transfer of moisture and nutrients, essential for their activation and growth.

The design of this system emphasizes the acceleration of fungal growth upon rehydration. The nonwoven scaffold, imbued with the encapsulated fungi, becomes an active site for digestion as soon as it encounters moisture. This immediate activation is particularly advantageous in absorbent products, where the presence of fluid triggers the fungi's emergence, thereby starting the digestion process without delay. The fungi, once activated, utilize the nonwoven material as a growth medium, rapidly expanding and initiating the digestion of the product.

It should be understood that the foregoing description is only illustrative of the various exemplary embodiments. Various alternatives and modifications can be devised by those skilled in the art without departing from the disclosure. Accordingly, the disclosure is intended to embrace all such alternatives, modifications and variances which fall within the scope of the disclosure.

1. An inoculant composition, comprising:
a first fungal species or strain of species;
a second fungal species or strain of species different from the first fungal species or strain of species, wherein
at least the first fungal species or strain of species is a saprophytic fungi,
each of the first fungal species or strain of species and second fungal species or strain of species is present in a concentration relative to each other and within the composition to allow inoculation on one or more polymer materials at an inoculation ratio of no greater than 1:1 fungal biomass to polymer material mass.

2. The inoculant of claim 1, wherein at least the first fungal species or strain of species is classified as a white rot fungal species.

3. The inoculant composition of claim 1, wherein each of the first fungal species or strain of species and second fungal species or strain of species is present in a concentration relative to each other and within the composition to allow inoculation on one or more polymer materials at an inoculation ratio of no greater than 0.5:1 fungal biomass to polymer material mass.

4. The inoculant composition of claim 1, wherein each of the first fungal species or strain of species and second fungal species or strain of species is present in a concentration relative to each other and within the composition to allow inoculation on one or more polymer materials at an inoculation ratio of no greater than 0.1:1 fungal biomass to polymer material mass.

5. The inoculant composition of claim 1, wherein the second fungal species or strain of species is classified as a brown rot fungal species or a soft rot fungal species.

6. The inoculant composition of claim 1, wherein the second fungal species or strain of species is classified as a white rot fungal species.

7. The inoculant composition of claim 1, wherein
the first fungal species or strain of species is an engineered fungal species or strain of species engineered to recognize plastic products as a food source and digest recalcitrant polymers via initiating enzymatic digestion more efficiently than a naturally occurring counterpart of the same fungal species or strain thereof, and
the second fungal species or strain of species is an engineered fungal species or strain of species engineered to recognize plastic products as a food source and digest recalcitrant long-chain carbons via initiating enzymatic digestion more efficiently than a naturally occurring counterpart of the same fungal species or strain thereof.

8. The inoculant composition of claim 6, wherein each of the first fungal species or strain of species and second fungal species or strain of species is engineered via a process to yield improved resilience to abiotic stress in the respective engineered fungal species or strains of species compared to a naturally occurring counterpart of the same fungal species or strain of species.

9. The inoculant composition of claim 1, wherein each of the first fungal species or strain of species and second fungal species or strain of species is engineered via a process to yield improved resilience to one or both of biotic and abiotic stress in the respective engineered fungal species or strains of species compared to a naturally occurring counterpart of the same fungal species or strain of species.

10. The inoculant composition of claim 1, further comprising an immobilization material, wherein each of the first fungal species or strain of species and second fungal species or strain of species is immobilized in the composition rendering the first engineered fungal species or strain of fungal species and second fungal species or strain of species dormant from metabolism.

11. (canceled)

12. (canceled)

13. The inoculant composition of claim 1, wherein one of the first fungal species or strain of species and second fungal species or strain of species is from phylum Basidiomycota.

14. The inoculant composition of claim 1, wherein the first fungal species or strain of species is from a different phyla of fungi than the second fungal species or strain of species.

15. The inoculant composition of claim 1, wherein the first fungal species or strain of species and second fungal species or strain of species are selected from a group consisting of Aureobasidium pullulans, Pestalotiopsis microspora, Aspergillus sp., Fusarium sp., Aspergillus versicolor, Aspergillus fumigatus, Pleurotus ostreatus, Pleurotus djamor, Dichomitus squalens, Trametes versicolor, Phanerochaete chrysosporium, Lentinula edodes, Phellinus pini, Inonotus obliquus and Fomitopsis spraguei.

16. (canceled)

17. The inoculant composition of claim 1, wherein one or both of the first fungal species or strain of species and second fungal species or strain of species is present within the composition in a form of spores of the first fungal species or strain of species.

18. The inoculant composition of claim 1, wherein one or both of the first fungal species or strain of species and second fungal species or strain of species is a filamentous fungi from any of the divisions of phylum fungi possessing a filamentous stage in their life cycle.

19. (canceled)

20. An inoculant composition, comprising:
a first engineered fungal species or strain of fungal species engineered to recognize plastic products as a food source and digest recalcitrant long-chain carbons more efficiently than a naturally occurring counterpart of the same fungal species or strain of species, wherein
the first engineered fungal species or strain of fungal species is immobilized within an immobilization material rendering the first engineered fungal species or strain of fungal species dormant from metabolism.

21. The inoculant composition of claim 20, wherein the engineered fungal species or strain of fungal species is engineered via a process of:
providing fungal mycelium to a growth media that includes an initial concentration of simple carbon and plastic material, and
reducing concentration of the simple carbon from the initial concentration over time while maintaining presence of plastic material within the media.

22. The inoculant composition of claim 21, wherein the first engineered fungal species or strain of fungal species is engineered for improved resilience to abiotic stress compared to a naturally occurring counterpart of the same fungal species or strain of species.

23. The inoculant composition of claim 20, wherein the first engineered fungal species or strain of fungal species is engineered for improved resilience to abiotic stress compared to a naturally occurring counterpart of the same fungal species or strain of species.

24. The inoculant composition of claim 20, wherein the immobilization material is configured to absorb moisture which thereby initiates mobilization of the first engineered fungal species or strain of fungal species from its immobilized state.

25. The inoculant composition of claim 22, wherein the immobilization material is configured to absorb moisture which thereby initiates mobilization of the first engineered fungal species or strain of fungal species from its immobilized state.

26. The inoculant composition of claim 21, wherein the immobilization material is configured to absorb moisture which thereby initiates mobilization of the first engineered fungal species or strain of fungal species from its immobilized state.

27. (canceled)

28. The inoculant composition of claim 20, wherein the first fungal species or strain of species is from one of phyla Basidiomycota and Ascomycota.

29. (canceled)

30. The inoculant composition of claim 20, wherein the first fungal species or strain of species is a filamentous fungi from any of the divisions of phylum fungi possessing a filamentous stage in their life cycle.

31. (canceled)

32. A method for transforming long chain carbon materials in plastic waste, comprising:
a) forming an inoculum comprising one or more preselected fungal species or preselected fungal strains or combination thereof capable of consuming long chain carbon molecules in polymer materials via metabolism;
b) providing a plastic substrate comprising long chain carbon molecules;
c) adding said inoculum to said plastic substrate to form an inoculum/substrate composite;
d) allowing said one or more fungal species or fungal strains or a combination thereof to digest said plastic substrate in said composite and thereby transform long chain carbon materials therein.

33-36. (canceled)

37. The method of claim 32, wherein
the inoculum is formed by immobilizing the one or more fungal species or fungal strains or combination thereof, and
the inoculum is activated by contact with moisture in the inoculum/substrate composite, thereby causing emergence of the one or more fungal species or fungal strains or combination thereof.

38. (canceled)

39. The method of claim 32, wherein the preselected fungal species or preselected fungal strains or combination thereof include filamentous fungi from any of the divisions of phylum fungi possessing a filamentous stage in their life cycle.

40-45. (canceled)

46. The method of claim 32, wherein the one or more preselected fungal species includes at least one species in phylum Basidiomycota.

47-132. (canceled)
